(19) United States
(12) Patent Application Publication
(10) Pub. No.: US 2024/0075126 A1
(43) Pub. Date: Mar. 7, 2024

(54) SARS-CoV-2 mRNA Vaccine and Preparation Method and Use Thereof

(71) Applicant: Shenzhen Rhegen Biotechnology Co., Ltd., Shenzhen City (CN)

(72) Inventor: Yong HU, Shenzhen City (CN)

(21) Appl. No.: 18/260,497

(22) PCT Filed: Apr. 29, 2022
(86) PCT No.: PCT/CN2022/090196

§ 371 Date: Aug. 8, 2023

(30) Foreign Application Priority Data
Jan. 27, 2022 (CN) 202210101841.6

(51) Int. Cl. A61K 39/215; C12N 7/00

(52) U.S. Cl. CPC A61K 39/215; C12N 7/00; A61K 2039/53

(57) ABSTRACT

Provided is a SARS-CoV-2 mRNA vaccine, and the preparation method and use thereof. The present invention provides an mRNA molecule capable of encoding a target polypeptide, wherein the target polypeptide comprises an NTD-RBD natural domain in the Spike (S) protein of SARS-CoV-2, and wherein the NTD-RBD natural domain comprises an NTD fragment and an RBD fragment, the NTD fragment and the RBD fragment being linked together via a natural amino acid sequence derived from the S protein as a linker. The present invention provides an mRNA encoding a NTD-RBD natural domain in the Spike protein of SARS-CoV-2, which achieves an immune effect against SARS-CoV-2 mutant strains and is widely applicable.

### TECHNICAL FIELD

The present invention relates to an mRNA vaccine and the preparation method and use thereof, more particularly, to an mRNA vaccine against SARS-CoV-2, including the prime strain and/or mutant strains, and the preparation method and use thereof.

### BACKGROUND

mRNA vaccine technology is useful as a fast and flexible technology platform to effectively deal with the threats of various emerging viruses. Therefore, mRNA vaccines are considered to be one of the most potential vaccines against SARS-CoV-2 (severe acute respiratory syndrome coronavirus-2). With the development and maturity of chemically modified mRNA and nano-lipid carrier technology, the advantages of mRNA in the research and development of various virus vaccines have become increasingly prominent.

Due to the specificity of the immune response, the sequence of target antigenic epitopes (including B cell epitopes and T cell epitopes) directly determines the type of immune response. Given that both B cell immunity and T cell immunity are indispensable in the clearance of the SARS-CoV-2 virus, the selection of a suitable immunogenic fragment is a key step in the design of new coronavirus vaccines. When designing target antigens for SARS-CoV-2 mutant strains, it is necessary to consider that some epitopes may induce non-neutralizing antibodies, which cannot play an effective protective function, but may rather cause an ADE effect and aggravate the degree of virus infection. Under these circumstances, therefore, further investigation needs to be conducted as regards whether the full-length S protein can continue to be used as a vaccine antigen. Upon studying of the serum of patients with COVID-19, it was found that the NTD and RBD fragments of the S protein contain multiple effective epitopes for B cells and T cells, and can induce potent protective antiviral immunity, which further proves the importance of epitope analysis when designing SARS-CoV-2 vaccines.

WO2021159040A9 discloses an mRNA vaccine encoding the NTD and RBD regions of the SARS-CoV-2 S protein. In designing the amino acid sequences, a glycine-serine linker was used to link the NTD and RBD regions.

On the other hand, new SARS-CoV-2 variants have been discovered, with a significant change in the prevalent strains around the world, and the immune escape thereof to various extents have brought challenges to the research and development of existing and future SARS-CoV-2 vaccines.

### SUMMARY OF INVENTION

An object of the present invention is to provide an mRNA vaccine against SARS-CoV-2.

Another object of the present invention is to provide a preparation method of the mRNA vaccine against SARS-CoV-2.

Yet another object of the present invention is to provide a DNA template for the mRNA vaccine against SARS-CoV-2.

Still another object of the present invention is to provide the use of the mRNA vaccine against SARS-CoV-2.

In an aspect, the present invention provides an mRNA molecule capable of encoding a target polypeptide, wherein the target polypeptide comprises the NTD-RBD natural domain in the Spike (S) protein of SARS-CoV-2, and wherein the NTD-RBD natural domain comprises an NTD fragment and an RBD fragment, and a natural amino acid sequence derived from the S protein is used as a linker linking the NTD fragment and the RBD fragment.

According to a particular embodiment of the invention, the mRNA molecule provided by the present invention further encodes a signal peptide at the N terminal of the NTD-RBD natural domain.

According to a particular embodiment of the invention, the amino acid sequence encoded by the mRNA molecule provided by the present invention comprises from the N terminal to the C terminal, in this order, a signal peptide, an NTD fragment, a linker, and an RBD fragment.

According to a particular embodiment of the invention, the signal peptide includes but not limited to: a sequence consisting of amino acids at position 1 to position 12 of SEQ ID NO: 1 (MFVFLVLLPLVS).

According to a particular embodiment of the invention, in the NTD-RBD natural domain encoded by the mRNA molecule provided by the present invention, the linker has an amino acid sequence of SEQ ID NO: 50.

According to a particular embodiment of the invention, in the NTD-RBD natural domain encoded by the mRNA molecule provided by the present invention, the NTD fragment has an amino acid sequence selected from:
(a) the amino acid sequence consisting of position 1 to position 289 of SEQ ID NO: 49;
(b) a sequence derived by substitution, addition and/or deletion of one or more amino acids from and having the same function as the amino acid sequence of (a).

According to a particular embodiment of the invention, in the NTD-RBD natural domain encoded by the mRNA molecule provided by the present invention, the RBD fragment has an amino acid sequence selected from:
(c) the amino acid sequence consisting of position 304 to position 526 of SEQ ID NO: 49;

(d) a sequence derived by substitution, addition and/or deletion of one or more amino acids from and having the same function as the amino acid sequence of (c).

In the present invention, said “same function” means it has the same immunogenicity.

According to a particular embodiment of the invention, the NTD-RBD natural domain encoded by the mRNA molecule provided by the present invention has an amino acid sequence as follows:
an amino acid sequence represented by any one of SEQ ID NO: 44 to SEQ ID NO: 49; or
a derivative amino acid sequence having an identity of 92.78% or more with respect to any one of SEQ ID NO: 44 to SEQ ID NO: 49.

According to a particular embodiment of the invention, the NTD-RBD natural domain encoded by the mRNA molecule provided by the present invention has an amino acid sequence of one of SEQ ID NO: 49, SEQ ID NO: 48, SEQ ID NO: 47, SEQ ID NO: 46, SEQ ID NO: 45, and SEQ ID NO: 44.

According to a particular embodiment of the invention, the amino acid sequence encoded by the mRNA molecule provided by the present invention is SEQ ID NO: 21, SEQ ID NO: 17, SEQ ID NO: 13, SEQ ID NO: 9, SEQ ID NO: 5, or SEQ ID NO: 1.

According to a particular embodiment of the invention, the protein-encoding region of the mRNA molecule provided by the present invention includes a sequence consisting of nucleotides at positions 37 to 1623 of SEQ ID NO: 25, a sequence consisting of nucleotides at positions 37 to 1623 of SEQ ID NO: 26, a sequence consisting of nucleotides at positions 37 to 1623 of SEQ ID NO: 27, a sequence consisting of nucleotides at positions 37 to 1614 of SEQ ID NO: 28, a sequence consisting of nucleotides at positions 37 to 1614 of SEQ ID NO: 29, a sequence consisting of nucleotides at positions 37 to 1614 of SEQ ID NO: 30, a sequence consisting of nucleotides at positions 37 to 1617 of SEQ ID NO: 31, a sequence consisting of nucleotides at positions 37 to 1617 of SEQ ID NO: 32, a sequence consisting of nucleotides at positions 37 to 1617 of SEQ ID NO: 33, a sequence consisting of nucleotides at positions 37 to 1623 of SEQ ID NO: 34, a sequence consisting of nucleotides at positions 37 to 1623 of SEQ ID NO: 35, a sequence consisting of nucleotides at positions 37 to 1623 of SEQ ID NO: 36, a sequence consisting of nucleotides at positions 37 to 1623 of SEQ ID NO: 37, a sequence consisting of nucleotides at positions 37 to 1623 of SEQ ID NO: 38, a sequence consisting of nucleotides at positions 37 to 1623 of SEQ ID NO: 39, a sequence consisting of nucleotides at positions 37 to 1614 of SEQ ID NO: 40, a sequence consisting of nucleotides at positions 37 to 1614 of SEQ ID NO: 41, or a sequence consisting of nucleotides at positions 37 to 1614 of SEQ ID NO: 42.

According to a particular embodiment of the invention, the protein-encoding region of the mRNA molecule provided by the present invention has a sequence represented by SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 42.

According to a particular embodiment of the invention, the mRNA molecule provided by the present invention is subjected to 1-methylpseuduridine modification.

According to a particular embodiment of the invention, the mRNA molecule provided by the present invention further comprises a 5′-UTR sequence and/or a 3′-UTR sequence.

In particular, a Kozak sequence may be or may be not included in the 5′-UTR sequence. In some preferred embodiments of the invention, the 5′-UTR has a sequence represented by SEQ ID NO: 51.

In some preferred embodiments of the invention, the 3′-UTR sequence has a sequence represented by SEQ ID NO: 52.

According to a particular embodiment of the invention, the mRNA molecule provided by the present invention is further subjected to 3′ tailing modification and/or at least one 5′ capping modification.

Particularly, the 3′ tailing modification includes a poly-A tail, the poly-A tail being a polyadenosinic acid with a linker inserted or not inserted therein.

Particularly, the cap structure in the at least one 5′ capping modification is selected from Cap0, Cap1, ARCA, inosine, N1-methyl-guanosine, 2′-flurorine-guanosine, 7-deaza-guanosine, 8-oxy-guanosine, 2-amino- guanosine, LNA- guanosine, or 2-azido-guanosine.

According to a particular embodiment of the invention, the mRNA molecule provided by the present invention is an isolated mRNA.

According to a particular embodiment of the invention, the mRNA molecule provided by the present invention is purified. The purification includes but not limited to chromatography, lithium chloride or ethanol precipitation, centrifuge column, chlorine extraction, ethanol precipitation or gel purification.

In another aspect, the present invention provides a DNA molecule encoding any of above-mentioned mRNA molecules according to the invention.

In yet another aspect, the present invention provides a recombinant plasmid comprising the above-mentioned DNA molecule according to the invention.

In still another aspect, the present invention provides a lipid nanoparticle supported with any of above-mentioned mRNA molecules according to the invention.

In another aspect, the present invention provides a pharmaceutical composition comprising any of above-mentioned mRNA molecules according to the invention and a pharmaceutically acceptable excipient. The excipient may be selected from solvent, aqueous solvent, non-aqueous solvent, dispersing medium, diluting agent, dispersing agent, suspension adjuvant, surfactant, isotonicity agent, thickener or emulsifier, preservative, lipid, lipidoid liposome, lipid nanoparticle, core-shell nanoparticle, polymer, lipoplexe peptide, protein, cell, hyaluronidase and the mixture thereof.

In yet another aspect, the present invention provides the use of the mRNA molecule, the DNA molecule, the recombinant plasmid, the lipid nanoparticle, or the pharmaceutical composition according to the invention in the manufacture of a SARS-CoV-2 mRNA vaccine.

In still another aspect, the present invention provides a SARS-CoV-2 mRNA vaccine comprising the mRNA molecule according to the invention. In some particular embodiments of the invention, the vaccine is in a dosage form of lipid nanoparticles.

According to a particular embodiment of the invention, in the SARS-CoV-2 mRNA vaccine according to the invention, the lipid nanoparticles have a particle size of 50 to 200 nm, preferably 50 to 150 nm.

According to a particular embodiment of the invention, in the SARS-CoV-2 mRNA vaccine according to the invention, the lipid nanoparticle comprises the mRNA and a lipid, and wherein the lipid includes:
a) one or more of a positively charged lipid and/or ionizable lipid;
b) a neutral auxiliary lipid;
c) cholesterol;
d) a PEGylated lipid.

According to a particular embodiment of the invention, in the SARS-CoV-2 mRNA vaccine according to the invention, the molar ratio of the nitrogen in the positively charged lipid and/or ionizable lipid to phosphorus in the mRNA is 5:1 to 20:1.

According to a particular embodiment of the invention, for the SARS-CoV-2 mRNA vaccine according to the invention, in the lipid nanoparticle, with respect to a total moles of the lipid of 100%, each lipid component is in a proportion by mole of:
the positively charged lipid and/or ionizable lipid, 46 to 50%;
the neutral auxiliary lipid, 5 to 10%;
cholesterol, 38.5 to 48%;
the PEGylated lipid, 0 to 3%.

According to a particular embodiment of the invention, for the SARS-CoV-2 mRNA vaccine according to the invention, in the lipid nanoparticle, the ionizable lipid includes but not limited to: one or more of (dilinoleyl)methyl 4-(N,N-dimethylamino)butanoate (Dlin-MC3-DMA), SM-102, and ((4-hydroxybutyl)azanediyl)bis(hexane-6,1-diyl)bis(2-hexyldecanoate) (ALC-0315); the positively charged lipid includes but not limited to: one or more of DOTMA and DOTAP; the neutral auxiliary lipid includes but not limited to: one or more of DSPC, DOPE, and DSPE; the PEGylated lipid includes but not limited to: one or more of methoxypoly(ethylene glycol) ditetradecylacetamide (ALC-0159) and DMG-PEG.

According to a particular embodiment of the invention, the SARS-CoV-2 mRNA vaccine according to the invention is in a lyophilized form or a frozen form.

In still another aspect, the present invention further provides a preparation method of a SARS-CoV-2 mRNA vaccine comprising a process of preparing the mRNA molecule. More particularly, the method comprises the steps of:
the DNA fragment encoding an NTD-RBD natural domain peptide fragment is synthesized, cloned to a plasmid as a template, and transcribed to prepare the target mRNA molecule;
preferably, the target mRNA molecule is any of the above-mentioned mRNA molecules according to the invention.

According to a particular embodiment of the invention, the present invention provides a preparation method of the mRNA molecule, wherein the process of synthesizing the DNA fragment encoding an NTD-RBD natural domain peptide fragment may be done by oneself or by commission. The DNA fragment is cloned to a plasmid as a template, and the target mRNA molecule may be prepared by reaction as follows:
a. three-step approach: an mRNA without capping and tailing is obtained upon in vitro transcription; a polyA structure (with a linker inserted or not inserted therein) is attached to the mRNA tail under the action of RNA polymerase and ATP, while a capping structure is attached to the 5′ terminal of the uncapped mRNA under the catalysis of a capping enzyme, so as to obtain a capped and tailed mRNA molecule;
b. two-step approach: a tailed mRNA without capping is obtained upon in vitro transcription; a capping structure is attached to the 5′ terminal of the uncapped mRNA under the catalysis of a capping enzyme, so as to obtain a capped and tailed mRNA molecule;
c. one-step approach: in vitro transcription and capping are carried out to obtain a capped and tailed mRNA molecule.

According to a particular embodiment of the invention, the preparation method of a SARS-CoV-2 mRNA vaccine according to the invention further comprises:
the prepared mRNA molecule is dissolved into an aqueous phase consisting of a citric acid buffer solution, and mixed with a lipid dissolved in an ethanol phase by using an impact jet flow method or a microfluidic method or the like, to prepare lipid nanoparticles having the mRNA supported thereon. Here, the lipid components may include, as mentioned above, ionizable phospholipids, neutral auxiliary phospholipids, cholesterol, PEGylated phospholipids and the like.

According to a particular embodiment of the invention, the preparation method of a SARS-CoV-2 mRNA vaccine according to the invention further comprises a process of making the prepared lipid nanoparticles into a frozen formulation or a lyophilized formulation.

According to a particular embodiment of the invention, in the preparation method of a SARS-CoV-2 mRNA vaccine according to the invention, the process of making the lipid nanoparticles into a lyophilized formulation comprises:
a) a buffer solution containing the lipid nanoparticles and a cryoprotectant is prepared;
b) the temperature is lowered for pre-freezing;
c) the temperature is raised under a vacuum condition, so as to dry the system to a moisture content of 3% or less, so as to prepare a dried formulation of the lipid nanoparticles.

According to a particular embodiment of the invention, in the preparation method of a SARS-CoV-2 mRNA vaccine according to the invention, the cryoprotectant includes but not limited to one or more of the following cryoprotectant 1 to cryoprotectant 3:
cryoprotectant 1: sucrose;
cryoprotectant 2: sucrose and non-ionic surfactant;
cryoprotectant 2: sucrose and trehalose.

According to a particular embodiment of the invention, in the preparation method of a SARS-CoV-2 mRNA vaccine according to the invention, in the prepared buffer solution containing the lipid nanoparticles and the cryoprotectant, sucrose is at a concentration (m/v) of 10 to 20%, i.e., 10 to 20 g/100 mL, preferably 12 to 18%; trehalose is at a concentration (m/v) of 0 to 20%, preferably 0 to 5%, more preferably 0.5 to 3%; the non-ionic surfactant is at a concentration (m/v) of 0 to 2%. According to some particular embodiments of the invention, the non-ionic surfactant includes but not limited to poloxamer, for example, may be Pluronic F-68. The concentration (m/v) of the poloxamer in the prepared buffer solution containing the lipid nanoparticles and the cryoprotectant is preferably 0 to 1%.

In the present invention, SEQ ID NO: 1, SEQ ID NO: 5, SEQ ID NO: 9, SEQ ID NO: 13, SEQ ID NO: 17, SEQ ID NO: 21 are the amino acid sequences of the NTD-RBD antigen of the SARS-CoV-2 prime strain, the Alpha strain, the Beta strain, the Gamma strain, the Delta strain, and the Omicron strain, respectively.

SEQ ID NO: 2, SEQ ID NO: 3, SEQ ID NO: 4 are the sequences of DNA fragments of the SARS-CoV-2 prime strain codon-optimized for the human, mouse, rat, respectively; SEQ ID NO: 25, SEQ ID NO: 26, SEQ ID NO: 27 are the sequences of the corresponding mRNA encoding regions.

SEQ ID NO: 6, SEQ ID NO: 7, SEQ ID NO: 8 are the sequences of DNA fragments of the SARS-CoV-2 Alpha strain codon-optimized for the human, mouse, rat, respectively; SEQ ID NO: 28, SEQ ID NO: 29, SEQ ID NO: 30 are the sequences of the corresponding mRNA encoding regions.

SEQ ID NO: 10, SEQ ID NO: 11, SEQ ID NO: 12 are the sequences of DNA fragments of the SARS-CoV-2 Beta strain codon-optimized for the human, mouse, rat, respectively; SEQ ID NO: 31, SEQ ID NO: 32, SEQ ID NO: 33 are the sequences of the corresponding mRNA encoding regions.

SEQ ID NO: 14, SEQ ID NO: 15, SEQ ID NO: 16 are the design of DNA fragments of the SARS-CoV-2 Gamma strain codon-optimized for the human, mouse, rat, respectively; SEQ ID NO: 34, SEQ ID NO: 35, SEQ ID NO: 36 are the sequences of the corresponding mRNA encoding regions.

SEQ ID NO: 18, SEQ ID NO: 19, SEQ ID NO: 20 are the design of DNA fragments of the SARS-CoV-2 Delta strain codon-optimized for the human, mouse, rat, respectively; SEQ ID NO: 37, SEQ ID NO: 38, SEQ ID NO: 39 are the sequences of the corresponding mRNA encoding regions.

SEQ ID NO: 22, SEQ ID NO: 23, SEQ ID NO: 24 are the design of the SARS-CoV-2 Omicron strain codon-optimized for the human, mouse, rat, respectively; SEQ ID NO: 40, SEQ ID NO: 41, SEQ ID NO: 42 are the sequences of the corresponding mRNA encoding regions.

SEQ ID NO: 43 is the mRNA sequence of the RBD antigen of the SARS-CoV-2 Delta strain.

SEQ ID NO: 44, SEQ ID NO: 45, SEQ ID NO: 46, SEQ ID NO: 47, SEQ ID NO: 48, SEQ ID NO: 49 are the amino acid sequences of the NTD-RBD natural domains of the prime strain, the Alpha strain, the Beta strain, the Gamma strain, the Delta strain, and the Omicron strain, respectively.

SEQ ID NO: 50 is the amino acid sequence of the linker in the NTD-RBD natural domain.

In the present invention, unless specifically indicated or clearly determined according to the context, the SARS-CoV-2 as mentioned includes the prime strain and/or mutant strains.

In some particular embodiments of the invention, an antigen epitope analysis is first conducted in the invention, and an mRNA vaccine formulation with NTD-RBD as the antigen target, LNP as the delivery carrier, and immunization by intramuscular injection is established. Meanwhile, by synthesizing the mRNA encoding the virus antigen fragment through UTR and codon optimization and by in vitro transcription methods, efficient expression of human cells are finally realized, with protection against all major mutant strains currently prevalent. The mRNA vaccine has a short overall production period, simply procedures and low production cost, may be stored for a long time and easily transported, without requiring a cold chain. It is impossible for traditional vaccination to quickly respond to public health events caused by many new viruses, whereas mRNA vaccines are more readily applicable, with flexible design of the sequence to cope with various pathogens, and play a critical role in fast research and development of quickly transcribed vaccines against acute infectious diseases.

The mRNA vaccine against the NTD-RBD antigen as designed in the present invention may elicit a more potent neutralizing antibody effect, given that the amount of vaccination is the same.

### BRIEF DESCRIPTION OF THE DRAWINGS

FIG. 1 shows the particle size and PDI test results of an mRNA-LNP prepared in a particular example according to the invention.

FIG. 2 shows the test results of the encapsulation percentage of an mRNA-LNP prepared in a particular example according to the invention.

FIG. 3 shows the appearance of an mRNA lyophilized vaccine prepared in a particular example according to the invention.

FIG. 4 illustrates the in vitro activity effects of an mRNA lyophilized vaccine prepared in a particular example according to the invention.

FIG. 5 illustrates the in vivo activity effects of an mRNA lyophilized vaccine prepared in a particular example according to the invention.

FIG. 6 shows the test results of the specific antibody titer of the mRNA vaccine according to the invention.

FIG. 7 shows a comparison of antigenicity between the RBD and NTD-RBD antigens.

FIG. 8 shows the cross protection effect against various strains of the NTD-RBD mRNA vaccine according to the invention.

FIG. 9 illustrates the strong cellular immunization induced by the NTD-RBD vaccine according to the invention.

FIG. 10 shows the experimental results of in vivo challenging in ACE2 mice with the NTD-RBD vaccine according to the invention.

FIG. 11 shows the experimental results of the NTD-RBD vaccine according to the invention as a booster dose.

FIG. 12 shows the experimental results of the relative expression amount of the protein expression induced by the mRNA according to certain particular examples according to the invention.

### DETAILED DESCRIPTION OF INVENTION

The present invention may be further described by means of the following examples, but the scope of the present invention is not limited to the following examples. Those skilled in the art will understand that various changes and modifications can be made in the present invention without departing from the spirit and scope of the invention.

The materials used in the tests and test methods are generally and/or specifically described in the invention. Although many of the materials and operation procedures used for the purposes of the present invention are known in the art, description as detailed as possible is herein provided in this invention. The operation procedures that are not described in details are performed according to the conventional procedures of the art in the related field or those recommended in the instructions provided by the manufacturer.

### EXAMPLE 1: mRNA VACCINE AND PREPARATION METHOD THEREOF

This example provides an mRNA vaccine, with the preparation method thereof mainly performed according to the following procedures.

1. A corresponding DNA fragment was synthesized according to the mRNA to be synthesized, and the DNA fragment was then cloned into an expression plasmid to obtain a recombinant plasmid. The recombinant plasmid was transfected into a host cell to obtain a recombinant cell, and the plasmid was extracted from the proliferated recombinant cells.

The constructed plasmid was subjected to enzyme digestion and linearization, with an enzyme digestion reaction system as follows (take the enzyme digestion of 2 μg recombinant plasmid as an example):
10×Digestion Buffer I 2μl
BspQI (10 U/μl) (purchased from Vazyme) 1μl
Plasmid DNA 2 μg
RNase-free Water up to 20 μl

The above enzyme digestion system was placed at 50° C. for 1 h. After the reaction was completed, 1 μl each of the system before and after the enzyme digestion was taken for DNA agarose gel electrophoresis (1.5% agarose gel, 5V/min, 40min). Comparison of the electrophoresis results showed whether the recombinant plasmid was enzymatically digested completely.

Eligibility criteria: a single band was present in electrophoresis assay; with respect to the supercoiled plasmid before enzyme digestion, the band was positioned above the supercoiled plasmid; the size met the expected demand.

Assay results: a single band was present; the size was as expected and the band was positioned above the supercoiled plasmid.

2. DNA template ultrafiltration

The DNA template obtained above was concentrated using a Millipore 30Kd ultrafiltration tube.

3. DNA template FPLC purification

To the above DNA concentrate obtained by ultrafiltration, an equal volume of a phenol/chloroform/isoamyl alcohol mixture (phenol/chloroform/isoamyl alcohol volume ratio=25/24/1) was added and sufficiently shaken before centrifugation at 12000 g for 15 min.

The precipitate was removed, and the supernatant was transferred to a new centrifuge tube, into which 3 M NaAc (pH 5.2) at a volume of 1/10 of that of the supernatant was added and evenly mixed, and anhydrous ethanol of a twice volume was then added and evenly mixed. The mixture was placed at −20° C. for 30 min.

Centrifugation was carried out at 4° C., 12000 g for 10 min, and the supernatant was discarded.

The precipitate was washed with 70% ethanol, centrifuged at 12,000 g for 5 min, and the supernatant was and air-dried on an ultra-clean bench for 5 min.

The purified DNA template was dissolved in an appropriate amount of RNase-free water.

The concentration of purified template, as well as the ratios of 260/280 and 260/230, were determined by NanoDrop. Samples were taken for DNA agarose gel electrophoresis assay (1.5% agarose, 5V/min, 40 min).

Eligibility Criteria: 260/280 between 1.8 and 2.1, 260/230 between 1.6 and 2.2.

Assay results: a concentration at 500 ng/μμl, 260/280=1.90, 260/230=1.7.

4. Template ultrafiltration after FPLC purification

The DNA template purified by FPLC was concentrated by using a Millipore 30Kd ultrafiltration tube, and eluted and dissolved with RNase-free water. The concentration of template after ultrafiltration and the ratios of 260/280 and 260/230 were determined by NanoDrop. Finally, it was diluted with RNase-free water to 150 ng/μl.

Assay results: a concentration at 15 ng/μl, 260/280=1.95, 260/230=1.85.

5. In vitro synthesis of mRNA

In vitro synthesis of the mRNA was performed in a thermostatic reactor.

The synthesis was carried out according to following synthesis system (reagents added in this order):

With a reaction volume of 1600 μl (i.e., the reaction volume of a single tube, placed in a 2 ml RNase-free tube; multiple tubes were reacted at the same time): RNA-free water 440 μl, 7.5 mM ATP 160 μl, 7.5 mM N1-methyl-pseudouridine 160 μl, 7.5 mM CTP 160 μl, 7.5 mM GTP 160 μl, 7.5 mM M7G(2′OMeA)pG 160 μl, 150 ng/μ1 DNA template 40 μl, 10×Buffer 160 μl and Enzyme Mix 160 μl.

The in vitro RNA synthesis procedure was carried out at 37° C. for 10 h.

After in vitro transcription and capping, the target mRNA molecules were obtained.

In each of the specific experimental examples of the present invention, in addition to the coding sequence, the target mRNA molecule further comprises 5′UTR (AGGGAGAUAAGAGAGAAAAGAAGAGUAAGAAGAAAUAUAAGAGCCGCCACC, SEQ ID NO: 51) and 3′UTR (GCUGCCUUCUGCGGGGCUUGCCUUCUGGCCAUGCCCUUCUUCUC UCCCUUGCACCUGUACCUCUUGGUCUUUGAAUAAAGCCUGAGUAGGAAG, SEQ ID NO: 52), with m7G+-5′-ppp-5′-Am2′-3′-p-(cap1) as the 5′CAP and a 3′ poly-A tail (SEQ ID NO: 53).

6. DNA template removal by DNase I digestion

120 μl of DNase I was added to each Tube after the in vitro mRNA synthesis.

Mixing by inversion was done 10 times before centrifuging at 1000 rpm for 10 s.

The solution was again placed in a thermostatic reactor at 37° C. for 1 h.

After the reaction was completed, the reaction solutions were combined into an RNase-free 50 ml tube, and residual DNA fragments were assayed. Results of three tests were 0.013 ng, 0.016 ng, 0.017 ng per 100 μg of mRNA.

7. Precipitation and recovery of mRNA

To each 50 ml Tube from the previous step, an equal volume of ammonium acetate solution was added.

The solution was mixed by inversion 10 times.

The mixture was placed at −20° C. for 2 h for precipitation.

Centrifugation was carried out at 17000 g, CC for 30 min.

The supernatant was removed, and the pellet was rinsed with 70% ethanol.

Centrifugation was carried out at 17000 g, CC for 10 min.

The 70% ethanol was removed, drying was conducted by evaporation on an ultra-clean bench, and 20 ml of RNase-free water was added to each tube.

After standing for 10 minutes, mixing was done by gentle blowing with a pipette tip.

The concentration of the recovered mRNA was 5 μg/μl by NanoDrop assay, with an A260/A280 of 1.90, and an A260/A230 of 2.0.

1 μl was taken and diluted 10 times, and RNA ScreenTape assay and agarose gel electrophoresis were conducted to determine the integrity of the fragment thereof.

Assay results: a matching band size and full fragment integrity.

8. mRNA purification by LiCI precipitation

To the mRNA recovered in the previous step, RNase-free water with a 1.5×volume thereof was added and evenly mixed.

A LiCI solution with a 1.5× volume of the mRNA pre-cooled at −20° C. was added thereto and evenly mixed.

The mixture was placed at −20° C. for 2 h.

Centrifugation was carried out at 16000 g for 20 min.

The supernatant was discarded, and the pellet was washed with 70% ethanol before centrifugation at 16,000 g for 15 min.

The supernatant was obtained and dried on an ultra-clean bench for 5 min.

The purified mRNA was dissolved in an appropriate amount of RNase-free water.

The purified mRNA was diluted to 2 μg/μ1 with 0.1 M citric acid.

9. LNP preparation

Aqueous phase preparation: mRNA was diluted in a citric acid buffer to a final concentration of 2 μg/μl.

The ethanol phase solution was prepared according to Table 1.

TABLE 1

| | Components required for a | Components required for a |
|---|---|---|
| Components | 1 mL formulation/μL | 0.5 mL formulation/μL |
| ALC-0315 | 559.7 | 279.8 |
| ALC-0159 | 117.2 | 58.6 |
| DSPC | 260.5 | 130.2 |
| Cholesterol | 62.7 | 31.3 |

PBS was prepared as an LNP diluent.

The operation steps of the syringe pump instrument were:

(1). Charging the A phase (mRNA buffer) into a 5 mL syringe, and charging the B phase (lipid compounds dissolved in absolute ethanol) into a 5 mL syringe, both of which were fastened on the syringe pump and tightly clamped;

(2). Connecting a chip to the syringe to set the flow rate of the syringe pump;

(3). Clicking the Start button of the syringe pump to inject the materials into the chip;

(4). Observing the color of the product at the outlet of the chip, discarding the first 5 milky white droplets (about 100 μl), and then starting to collect into 60 mL of PBS;

(5). Gently mixing the collected product by inversion before storing at 4° C.;

(6). Taking 0.3 mL as a sample for encapsulation rate test;

(7). Lysis: to 0.1 mL from step (6), adding 2μL of 10% Triton X-100 and mixing evenly, followed by incubation at room temperature for 10 min;

(8). Encapsulation rate test: diluting 64 μl of each of the samples from step (6) and step (7) by 5 times, as LNP RNA samples before and after lysis; determining the RNA concentration; calculating the encapsulation rate by dividing the concentration difference before and after lysis by the concentration after lysis;

(9) Conducting particle size and PDI tests and Zeta potential analysis on the Zetasizer nano instrument from Malvern Company by using standard testing methods, with a loading volume of 600 μl, DTS1070 as the sample cell, and a testing temperature of 25° C.

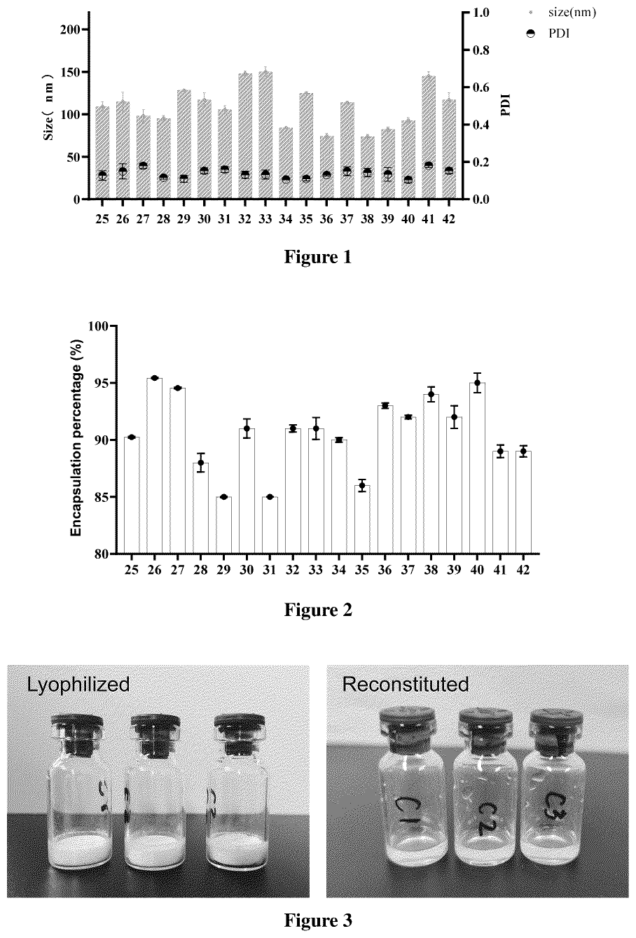

The test results of particle size, PDI and encapsulation rate of the mRNA-loaded LNPs prepared in this example are shown in FIGS. 1 and 2. In FIGS. 1 and 2, the numbers on the horizontal cordinate correspond to the sequence numbers of respective mRNAs. That is, sample 25 in the Figures corresponds to the mRNA sample of SEQ ID NO: 25, sample 26 in the Figures corresponds to the mRNA sample of SEQ ID NO: 26, sample 27 in the Figures corresponds to the mRNA sample of SEQ ID NO: 27, and so on; and the sample corresponding to 42 in the figure is the mRNA sample of SEQ ID NO: 42.

10. LNP lyophilization

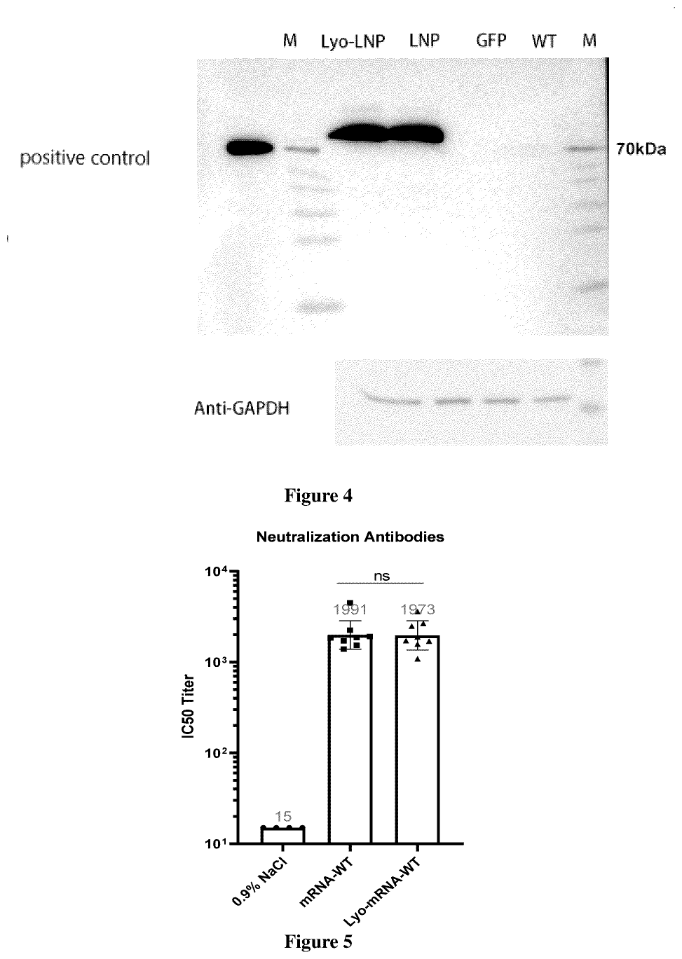

An cryoprotectant was added to the LNP solution and put into a lyophilizer, with a mass volume fraction (w/v%) of 15% sucrose and 2% trehalose. After pre-freezing with a cold trap (Xinzhi Scientz-10N) for 4 hours, the vacuum was turned on for vacuum suction for 48 hours, and then the samples were transferred to the upper layer of the lyophilizer for secondary drying for 16 hours. The sample temperature probe showed a temperature of about −30° C. during the drying in the cold trap, and the sample temperature probe showed a temperature of about 4° C. during the drying on the upper layer. The product (i.e., LNP lyophilized powder) was collected after drying, reconstituted with ultrapure water in a volume equal to that of the solution before lyophilization. The lyophilized powder quickly dissolved after the addition of ultrapure water in no more than 20 s during the whole process. RiboGreen™ was used to measure the encapsulation rate of the nanoparticles, and the particle size and zeta potential of the nanoparticles were measured by Omec NS-90Z Nanoparticle Size and Potential Analyzer. The mRNA lyophilized vaccine according to the present example was thus prepared. The picture of the finished product of the vaccine thus prepared and its appearance after reconstitution are shown in FIG. 3. 293T cells were culture by incubation with 2 μg of the mRNA-LNP solution, the lyophilized solution and GFP mRNA-LNP of 37# (corresponding to the mRNA sample of SEQ ID NO: 37), respectively. After 24 hours, the cell pellet was obtained followed by cell lysis, and the soluble proteins were harvested for Western blotting to determine the protein expression efficiency. In addition, 100 μg of the mRNA-LNP powder of 37# (corresponding to the mRNA sample of SEQ ID NO: 37) was reconstituted with 200 μl water for injection before inoculation; 6-week-old balb/c mice were inoculated twice, on day 1 and day 14, and the mouse serum was collected on day 35 to measure the anti-S protein-specific antibody titers in the serum. The results are shown in FIGS. 4 and 5. The lyophilization process essentially has no impact on the biological activity of the mRNA lipid particles.

### EXAMPLE 2

In this example, 100 μg mRNA-LNP powders of 25# to 42# (i.e., corresponding to the mRNA samples of SEQ ID NO: 25 to SEQ ID NO: 42, respectively) were each reconstituted with 200 μl water for injection before inoculation. 6-week-old balb/c mice were inoculated twice, on day 1 and day 14, and the mouse serum was collected on day 35 to measure the anti-S protein-specific antibody titers in the serum. Specifically, the following procedures were carried out:

1. Coating: the S1 protein (Yidiao Shenzhou, 40591-MM43) was diluted with a coating buffer to a solution of 200 ng/ml and added to a microtiter plate, with a volume of 100 μl in each well, and triplicated in three wells for each dilution. After covering with a sealing film, the plate was set at CC overnight.

2. Plate washing: after the 96-well plate was coated, the coating solution was poured onto an absorbent paper, and the plated was forcefully patted on the paper until there was no residue in the wells. A washing buffer was prepared, diluted 50× with deionized water, and added to the loading bottle of a plate washer. A program was set with a washing volume of 300 μl for each well, and washing was repeatedly carried out for 4 times.

3. Blocking: The washed plate was patted to dry the solution inside, and a Blocking buffer was added in a volume of 250 μl per well. Then, the plate was sealed with a sealing film, and blocking was carried out at room temperature for 2 hours.

4. Plate washing: The plate after blocking was washed as in step 2.

5. Serum incubation: Mouse serum was diluted 40×, 400×, 4000×, 40000×, 400000×, 4000000×, 40000000× with a dilution buffer, and added in a volume of 100 μl per well to the washed 96-well plate, and the plated was sealed with a sealing film and incubated at room temperature for 1.5 h.

6. Plate washing: Plate washing was carried out as in step 2, except increasing the washing number to 6 times.

7. Secondary antibody addition: HRP-labeled goat anti-mouse IgG was diluted with a dilution buffer by a dilution factor of 10000×, the diluted antibody was added to the plate in a volume of 100 μl per well, and the plated was sealed with a sealing film and incubated at room temperature in the dark 1 h.

8. Plate washing: Plate washing was carried out as in step 2. In this step, extra care was taken to completely clean the plate to be solution-free.

9. Color development: 100 μl of a TMB buffer was added, and color development was carried out in the dark for 20-30 minutes. At this time, positive samples appeared in blue.

10. Termination: 100 μl of a stop buffer was added, and the microtiter plate was read within 10 minutes, with the absorption wavelength set to 450 nm.

A standard curve was drawn based on the well absorption values of the standard, and a correlation coefficient of linear regression of greater than 0.0995 was required. The residual content of the S protein-specific antibody in the sample was calculated according to the absorbance of the product to be tested.

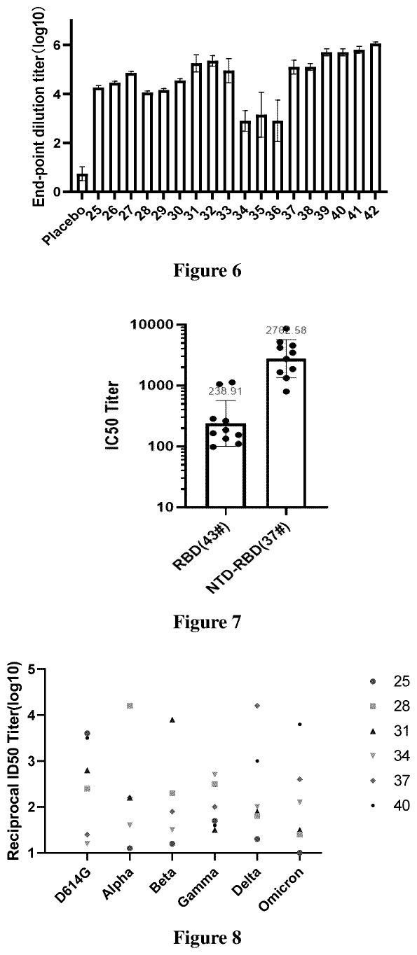

The results of the specific antibody titer assay are shown in FIG. 6. The results suggested that the NTD-RBD-encoding mRNA candidate vaccine had good immunogenicity and induced the production of specific antibodies against the S protein. The mice in the control group injected with saline were not able to produce a specific antibody against the S protein.

### EXAMPLE 3

In this example, the antibody titers in the RBD-encoding mRNA vaccine (43#, i.e., the sample corresponding to SEQ ID NO: 43) and the NTD-RBD-encoding vaccine (37#, i.e., the sample corresponding to SEQ ID NO: 37) were investigated through experiments. Specifically, the following procedures were carried out:

For vaccination, 6-8 weeks old female mice were inoculated with 5 μg of the SARS-CoV-2 mRNA vaccine (dissolved in PBS, 200 μl, intramuscular injection) on day 0 (0 d) and day 14 (14 d) respectively; on day 28 (28 d), the peripheral blood of the mice was sampled.

Vero E6 cells (24-well plate) were prepared, with serum inactivation by heating and serial dilutions in 3-fold. 100 μl of the serum and 100 μl of the virus stock solution (100 PFU) were mixed in equivalent volumes, and 100 μl of DMEM (containing 2% FBS) was mixed with an equivalent volume of 100 μl of the virus solution as a negative control. Incubation was carried out at 37° C. for 1 h.

The above serum-virus mixture (200 μl in total) was transferred to the Vero E6 cells in a 24-well plate and adsorbed for 1 h. During this time, gentle mixing could be carried out 3-4 times. The adsorption solution from the previous step was then removed, and the medium was changed to a methylcellulose medium, followed by incubation for 3 days. The system was fixed with paraformaldehyde, stained with crystal violet, and the number of plaques was then counted. Serum neutralization percentage was calculated based on the negative control. The curve was fitted by using the JMP analysis software with the Probit approach, and the PRNT₅₀ value was calculated.

The experimental results are shown in FIG. 7. The neutralizing antibody titer induced by NTD-RBD was higher than that of RBD, and consequently the immunogenicity of NTD-RBD was better than that of RBD.

### EXAMPLE 4

In this example, the cross-immune protection of the NTD-RBD mRNA vaccine against the 2019-nCoV pseudovirus was investigated through experiments. Specifically, the following procedures were carried out:

For vaccination, 6-8 weeks old female mice were inoculated with 5 μg of the SARS-CoV-2 mRNA vaccine (dissolved in PBS, 200 μl, intramuscular injection) on day 0 (0 d) and day 14 (14 d) respectively; on day 28 (28 d), the peripheral blood of the mice was sampled. A micro-neutralization assay was used to test the heat-inactivated serum, to determine the level of antibody neutralization of the 2019-nCoV pseudovirus infecting ACE2-expressing monolayer cells. 4 wells were diluted in a 96-well plate, and the viral cytopathic effect (cpe) in the cells was tested on day 3 and day 4. The degree of dilution of the serum (as assayed by the 50% end-point method) at which complete suppression of cpe was achieved in the serum was calculated by the Reed Muench equation. Nonparametric two-tailed t-test (Mann-Whiteny) was used for statistical analysis.

The experimental results are shown in FIG. 8 (the reference numbers in each panel of the figure were the samples corresponding to the same SEQ ID NOs., respectively). The mRNA vaccines constructed in the present invention had a cross-protection effect to various extents on the SARS-CoV-2 mutant strain.

### EXAMPLE 5: INDUCTION of STRONG CELLULAR IMMUNITY by NTD-RBD SARS-COV-2 VACCINES

In this example, the effect of the NTD-RBD SARS-CoV-2 vaccines in inducing cellular immunity was investigated. Specifically, the following procedures were carried out:
1. 96-well plate coating

PVDF membrane in a 96-well plate was infiltrated with 70% ethanol for 30 s.

A capture antibody (diluted in PBS) was added and kept at 4° C. overnight.

The plate was emptied of the coating solution, gently patted dry on paper, and washed with PBS (use of a plate washer was not allowed).

100 μl of 2% non-fat dry milk powder (or BSA) was added for incubation at room temperature for 2 hours to block the blank wells in the plate.

The plate was washed once with PBS.
2. Cell Stimulation and cytokine capture

For vaccination, 6-8 weeks old female mice were inoculated with 5 μg of the SARS-CoV-2 mRNA vaccine RH109(40#) (dissolved in PBS, 200 intramuscular injection) on day 0 (0 d) and day 14 (14 d) respectively; on day 28 (28 d), the peripheral blood of the mice was sampled. PBMCs were separated from fresh blood with Ficoll and counted, and the cells were diluted with a medium and added to a 96-well plate. The general number of cells used was 1 to 2×10⁵/well.

The 96-well plate was incubated overnight in a 37° C. CO₂ incubator. Moving or shaking the plate was not allowed.

An S protein stimulating material (pepmix) was added to the plate and incubated for 8 hours.

Incubation was carried out with PBS containing 0.1% Tween 20 for 10 minutes before the cells and unbound cytokines were removed. The plate was then washed 3 times with PBS containing 0.1% Tween 20.
3. Detection by Adding Detecting Antibody

A labeled detecting antibody (diluted in PBS containing 1% BSA) was added and incubated at room temperature for 1-2 hours.

A substrate was added for color developing (before adding the substrate, both sides of the plate membrane were washed with distilled water to avoid a background resulted from leaked solution). The formation of spots was monitored, and the reaction was terminated at an appropriate point.

The reaction was terminated by washing with distilled water.
4. Result Analysis

The 96-well plate was dried (the plate was kept in the dark at 4° C. overnight so that the edge of the spots were sharp and easier to be distinguished).

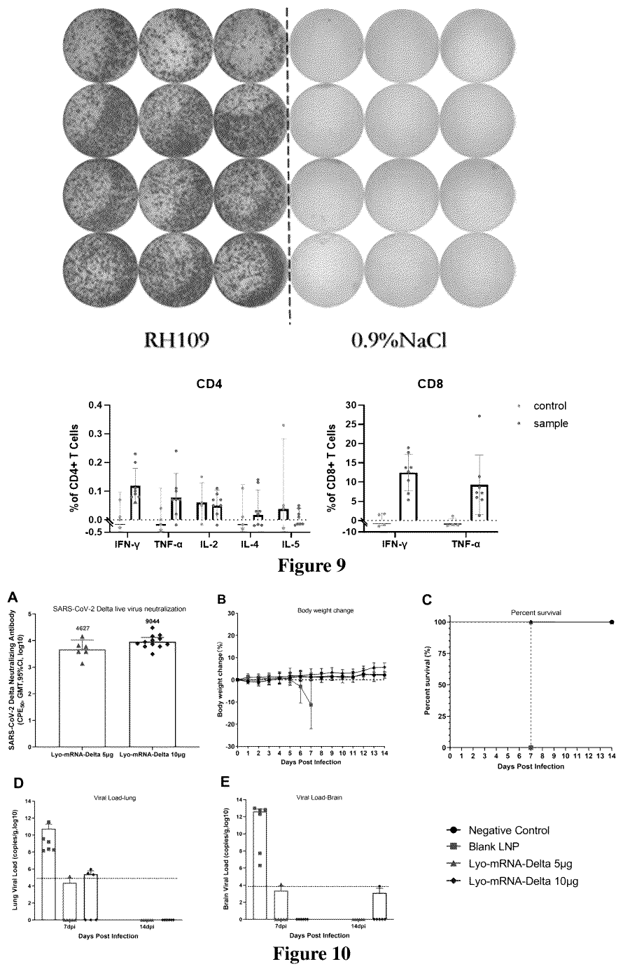

The results were analyzed by using a plate reader, as shown in FIG. 9. The ELISPot experiment showed that after the polypeptide stimulation, a large number of IFN-γ plaques were produced for the vaccinated mouse group up to 3000 plaques/1 million cells, while almost no plaque produced for the saline group, indicating that RH109 induced a strong cellular immune response.

Further analysis of CD4 and CD8 cells by flow cytometry intracellular staining showed that IL2⁺/TNF-α/IFN-γ⁺CD4⁺cells and TNF-α⁺/IFN-γ⁺CD8⁺cells were significantly increased, indicating that the RH109 vaccine activated strong Th1-type and CD8 killer T-cell immune responses.

### EXAMPLE 6

For vaccination, 6-8 weeks old female mice were inoculated with 5 μg of the SARS-CoV-2 mRNA vaccine (37#) (dissolved in PBS, 200 μl, intramuscular injection) on day 0 (0 d) and day 14 (14 d) respectively; ivD being the challenge period, and ivD0 was day 0 of the challenge. hACE2 transgenic mice were intranasally infected by SARS-CoV-2 (the Delta strain), and the infection dose was preliminarily 105 PFU. D42 is the duration of the challenge. After the challenge at ivD3 and ivD5, animals were euthanized in batches. Lungs (right lung) and other tissues and organs were harvested from the euthanized animals, RNA was extracted, and the viral load or live virus titer was determined by Q-PCR method. During the procedure, observation was made twice a day, and the body weight was measured once a week. The viral load and survival profile are shown in FIG. 10. It can be seen from FIG. 10 that the viral load in the lungs of the vaccinated mice was significantly lower than those of the control group after the true virus challenge experiment. The mice in the adjuvant control group all reached the criterion for euthanasia on day 7 after the challenge. Compared with the adjuvant control group, all the mice in the low-dose vaccine group and the high-dose vaccine group survived, indicating that the vaccine had a significant protective effect on the mice infected with the SARS-CoV-2 Delta strain.

### EXAMPLE 7

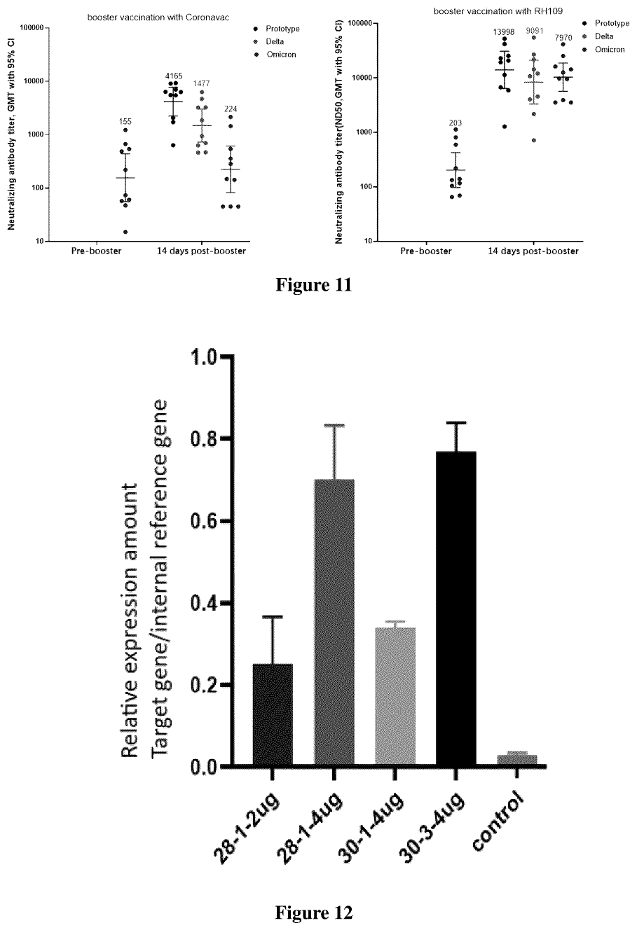

For vaccination, 6-8 weeks old female mice were inoculated with two doses of the SINOVAC inactivated vaccine on day 0 (0 d) and day 14 (14 d), and 5 μg of the SARS-CoV-2 mRNA vaccine RH109 (dissolved in PBS, 200 μl, intramuscular injection) on day 42 (42 d). On day 56 (56 d), the peripheral blood of the mice was sampled. A micro-neutralization assay was used to test the heat-inactivated serum, to determine the level of antibody neutralization of the SARS-CoV-2 pseudovirus infecting ACE2-expressing monolayer cells. 4 wells were diluted in a 96-well plate, and the viral cytopathic effect (cpe) in the cells was tested on day 3 and day 4. The degree of dilution of the serum (as assayed by the 50% end-point method) at which complete suppression of cpe was achieved in the serum was calculated by the Reed Muench equation. Nonparametric two-tailed t-test (Mann-Whiteny) was used for statistical analysis. As shown in FIG. 11, the RH109-immunized serum produced a high level of neutralizing antibodies to Omicron, Delta, and the prime strain, with a geometric average titer of 7970, 9091, and 13998, respectively. Among them, the tier was increased 39 times than that after the second dose against Omicron. Further, the titer was 35, 6.2, and 3.4 times against Omicron, Delta, and the prime strain respectively, as compared to the case where the SINOVAC inactivated vaccine was used as the third dose for boosting, indicating that RH109 was very suitable as a booster dose in addition to a first dose of an inactivated vaccine. Furthermore, various mRNAs according to the invention at different doses in inducing protein expression were also compared in the present invention through experiment; in this experiment, 293T cells were incubated with 2 μg or 4 μg of an mRNA-LNP solution of SEQ ID NO: 28, SEQ ID NO: 54, and SEQ ID NO: 55 respectively, the cell pellet was collected after 24 hours and then lysed, and the obtained bands of the target protein were assayed in parallel by grayscale analysis for the relative amount of intracellular protein expression within 24 hours. The results are shown in FIG. 12 (in this figure, reference number 28-1 corresponds to SEQ ID NO: 28, 30-1 corresponds to SEQ ID NO: 54, and 30-3 corresponds to SEQ ID NO: 55). The mRNA of the present invention showed excellent efficiency in protein expression.

It should be noted that those described above are merely preferred examples of the present invention, and are not intended to limit the present invention. Those skilled in the art would understand that various modifications and changes are available to the present invention. Any changes, equivalent substitution, modification and the like made within the spirit and principle of the present invention are intended to be included within the scope of the present invention.

1-31. (canceled)

32. An mRNA molecule encoding an amino acid sequence as shown in SEQ ID NO: 21.

33. The mRNA molecule according to claim 32, wherein the coding sequence of the mRNA is as shown in SEQ ID NO: 40, SEQ ID NO: 41, or SEQ ID NO: 42.

34. The mRNA molecule according to claim 32, wherein the mRNA has been subjected to 1-methylpseudouridine modification.

35. The mRNA molecule according to claim 32, further comprising a 5′-UTR sequence and/or a 3′-UTR sequence.

36. The mRNA molecule according to claim 35, wherein a Kozak sequence is or is not included in the 5′-UTR sequence.

37. The mRNA molecule according to claim 35, wherein the 5′-UTR has a sequence represented by SEQ ID NO: 51.

38. The mRNA molecule according to claim 35, wherein the 3′-UTR sequence has a sequence represented by SEQ ID NO: 52.

39. The mRNA molecule according to claim 35, wherein the mRNA has been subjected to a 3′ tailing modification and/or at least one 5′ capping modification.

40. The mRNA molecule according to claim 39, wherein the 3′ tailing modification includes a poly-A tail, and the poly-A tail is a polyadenosinic acid with or without a linker inserted therein;
the cap structure in the at least one 5′ capping modification is selected from Cap0, Cap1, ARCA, inosine, N1-methyl-guanosine, 2′-flurorine-guanosine, 7-deaza-guanosine, 8-oxy-guanosine, 2-amino-guanosine, LNA-guanosine, or 2-azido-guanosine.

41. The mRNA molecule according to claim 32, which is an isolated mRNA.

42. The mRNA molecule according to claim 41, the mRNA molecule is purified.

43. A SARS-CoV-2 mRNA vaccine comprising the mRNA molecule according to claim 32, wherein the vaccine is in a dosage form of lipid nanoparticles.

44. The SARS-CoV-2 mRNA vaccine according to claim 43, wherein the lipid nanoparticle comprises the mRNA and a lipid, and the lipid includes:
a) one or more of a positively charged lipid and/or ionizable lipid;
b) a neutral auxiliary lipid;
c) cholesterol; and
d) a PEGylated lipid.

45. The SARS-CoV-2 mRNA vaccine according to claim 44, wherein the molar ratio of the nitrogen in the positively charged lipid and/or ionizable lipid to phosphorus in the mRNA is 5:1 to 20:1.

46. The SARS-CoV-2 mRNA vaccine according to claim 44, wherein the ionizable lipid includes, but is not limited to, one or more of (dilinoleyl)methyl 4-(N,N-dimethylamino)butanoate, SM-102, and ((4-hydroxybutyl)azanediyl)bis(hexane-6,1-diyl)bis(2-hexyldecanoate);
the positively charged lipid includes, but is not limited to, one or more of DOTMA and DOTAP;
the neutral auxiliary lipid includes, but is not limited to, one or more of DSPC, DOPE, and DSPE; and
the PEGylated lipid includes, but is not limited to, one or more of methoxypoly(ethylene glycol) ditetradecylacetamide and DMG-PEG.

47. The SARS-CoV-2 mRNA vaccine according to claim 43, wherein the dosage form of the vaccine is a lyophilized form or a frozen form.

48. A method for preparing a SARS-CoV-2 mRNA vaccine, comprising the steps of:
synthesizing a DNA fragment encoding an NTD-RBD natural domain peptide, cloning it to a plasmid as a template, and transcribing it to prepare the target mRNA molecule;
wherein the target mRNA molecule is the mRNA molecule according to claim 32.

49. The method according to claim 48, further comprising:
dissolving the prepared mRNA molecule in an aqueous phase consisting of a citric acid buffer solution, and mixing it with lipid components dissolved in an ethanol phase by using an impact jet flow method or a microfluidic method or the like, to prepare lipid nanoparticles carrying the mRNA.

50. The method according to claim 49, further comprising a process of making the prepared lipid nanoparticles into a frozen formulation or a lyophilized formulation.

51. The method according to claim 50, wherein the process of making the lipid nanoparticles into a lyophilized formulation comprises:
a) preparing a buffer solution containing the lipid nanoparticles and a cryoprotectant;
b) lowering the temperature for pre-freezing; and
c) raising the temperature under a vacuum condition to dry the system to a moisture content of 3% or less, so as to prepare a dried formulation of the lipid nanoparticles.


SEQUENCE LISTING (55 sequences)

SEQ ID NO: 1 - protein, 541 aa, Artificial Sequence
  Antigen
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 2 - DNA, 1623 bp, Artificial Sequence
  For the preparation of mRNA vaccines
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttc

SEQ ID NO: 3 - DNA, 1623 bp, Artificial Sequence
  For the preparation of mRNA vaccines
atgttcgtgttcctggtgctgctgcctctggtgagcagccagtgcgtgaacctgaccaccagaacccagctgcctcctgcctacaccaacagcttcaccagaggcgtgtactaccctgacaaggtgttcagaagcagcgtgctgcacagcacccaggacctgttcctgcctttcttcagcaacgtgacctggttccacgccatccacgtgagcggcaccaacggcaccaagagattcgacaaccctgtgctgcctttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccctttcctgggcgtgtactaccacaagaacaacaagagctggatggagagcgagttcagagtgtacagcagcgccaacaactgcaccttcgagtacgtgagccagcctttcctgatggacctggagggcaagcagggcaacttcaagaacctgagagagttcgtgttcaagaacatcgacggctacttcaagatctacagcaagcacacccctatcaacctggtgagagacctgcctcagggcttcagcgccctggagcctctggtggacctgcctatcggcatcaacatcaccagattccagaccctgctggccctgcacagaagctacctgacccctggcgacagcagcagcggctggaccgccggcgccgccgcctactacgtgggctacctgcagcctagaaccttcctgctgaagtacaacgagaacggcaccatcaccgacgccgtggactgcgccctggaccctctgagcgagaccaagtgcaccctgaagagcttcaccgtggagaagggcatctaccagaccagcaacttcagagtgcagcctaccgagagcatcgtgagattccctaacatcaccaacctgtgccctttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccctaccaagctgaacgacctgtgcttcaccaacgtgtacgccgacagcttcgtgatcagaggcgacgaggtgagacagatcgcccctggccagaccggcaagatcgccgactacaactacaagctgcctgacgacttcaccggctgcgtgatcgcctggaacagcaacaacctggacagcaaggtgggcggcaactacaactacctgtacagactgttcagaaagagcaacctgaagcctttcgagagagacatcagcaccgagatctaccaggccggcagcaccccttgcaacggcgtggagggcttcaactgctacttccctctgcagagctacggcttccagcctaccaacggcgtgggctaccagccttacagagtggtggtgctgagcttcgagctgctgcacgcccctgccaccgtgtgcggccctaagaagagcaccaacctggtgaagaacaagtgcgtgaacttc

SEQ ID NO: 4 - DNA, 1623 bp, Artificial Sequence; identical to SEQ ID NO: 2
  For the preparation of mRNA vaccines

SEQ ID NO: 5 - protein, 538 aa, Artificial Sequence
  Antigen
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNF

SEQ ID NO: 6 - DNA, 1614 bp, Artificial Sequence
  For the preparation of mRNA vaccines
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatcagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccacctacggcgtgggctaccagccctacagagtggtggtgctgagcttcgagctgctgcacgcccccgccaccgtgtgcggccccaagaagagcaccaacctggtgaagaacaagtgcgtgaacttc

SEQ ID NO: 7 - DNA, 1614 bp, Artificial Sequence
  For the preparation of mRNA vaccines
atgttcgtgttcctggtgctgctgcctctggtgagcagccagtgcgtgaacctgaccaccagaacccagctgcctcctgcctacaccaacagcttcaccagaggcgtgtactaccctgacaaggtgttcagaagcagcgtgctgcacagcacccaggacctgttcctgcctttcttcagcaacgtgacctggttccacgccatcagcggcaccaacggcaccaagagattcgacaaccctgtgctgcctttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccctttcctgggcgtgtaccacaagaacaacaagagctggatggagagcgagttcagagtgtacagcagcgccaacaactgcaccttcgagtacgtgagccagcctttcctgatggacctggagggcaagcagggcaacttcaagaacctgagagagttcgtgttcaagaacatcgacggctacttcaagatctacagcaagcacacccctatcaacctggtgagagacctgcctcagggcttcagcgccctggagcctctggtggacctgcctatcggcatcaacatcaccagattccagaccctgctggccctgcacagaagctacctgacccctggcgacagcagcagcggctggaccgccggcgccgccgcctactacgtgggctacctgcagcctagaaccttcctgctgaagtacaacgagaacggcaccatcaccgacgccgtggactgcgccctggaccctctgagcgagaccaagtgcaccctgaagagcttcaccgtggagaagggcatctaccagaccagcaacttcagagtgcagcctaccgagagcatcgtgagattccctaacatcaccaacctgtgccctttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccctaccaagctgaacgacctgtgcttcaccaacgtgtacgccgacagcttcgtgatcagaggcgacgaggtgagacagatcgcccctggccagaccggcaagatcgccgactacaactacaagctgcctgacgacttcaccggctgcgtgatcgcctggaacagcaacaacctggacagcaaggtgggcggcaactacaactacctgtacagactgttcagaaagagcaacctgaagcctttcgagagagacatcagcaccgagatctaccaggccggcagcaccccttgcaacggcgtggagggcttcaactgctacttccctctgcagagctacggcttccagcctacctacggcgtgggctaccagccttacagagtggtggtgctgagcttcgagctgctgcacgcccctgccaccgtgtgcggccctaagaagagcaccaacctggtgaagaacaagtgcgtgaacttc

SEQ ID NO: 8 - DNA, 1614 bp, Artificial Sequence
  For the preparation of mRNA vaccines
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatcagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccacctacggcgtgggctaccagccctacagggtggtggtgctgagcttcgagctgctgcacgcccccgccaccgtgtgcggccccaagaagagcaccaacctggtgaagaacaagtgcgtgaacttc

SEQ ID NO: 9 - protein, 539 aa, Artificial Sequence
  Antigen
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 10 - DNA, 1617 bp, Artificial Sequence
  For the preparation of mRNA vaccines
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgccaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgaggggcctgccccagggcttcagcgccctggagcccctggtggacctgcccatcggcatcaacatcaccaggttccagaccctgctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaacatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtgaagggcttcaactgctacttccccctgcagagctacggcttccagcccacctacggcgtgggctaccagccctacagggtggtggtgctgagcttcgagctgctgcacgcccccgccaccgtgtgcggccccaagaagagcaccaacctggtgaagaacaagtgcgtgaacttc

SEQ ID NO: 11 - DNA, 1617 bp, Artificial Sequence
  For the preparation of mRNA vaccines
atgttcgtgttcctggtgctgctgcctctggtgagcagccagtgcgtgaacctgaccaccaggacccagctgcctcctgcctacaccaacagcttcaccaggggcgtgtactaccctgacaaggtgttcaggagcagcgtgctgcacagcacccaggacctgttcctgcctttcttcagcaacgtgacctggttccacgccatccacgtgagcggcaccaacggcaccaagaggttcgccaaccctgtgctgcctttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccctttcctgggcgtgtactaccacaagaacaacaagagctggatggagagcgagttcagggtgtacagcagcgccaacaactgcaccttcgagtacgtgagccagcctttcctgatggacctggagggcaagcagggcaacttcaagaacctgagggagttcgtgttcaagaacatcgacggctacttcaagatctacagcaagcacacccctatcaacctggtgaggggcctgcctcagggcttcagcgccctggagcctctggtggacctgcctatcggcatcaacatcaccaggttccagaccctgctgcacaggagctacctgacccctggcgacagcagcagcggctggaccgccggcgccgccgcctactacgtgggctacctgcagcctaggaccttcctgctgaagtacaacgagaacggcaccatcaccgacgccgtggactgcgccctggaccctctgagcgagaccaagtgcaccctgaagagcttcaccgtggagaagggcatctaccagaccagcaacttcagggtgcagcctaccgagagcatcgtgaggttccctaacatcaccaacctgtgccctttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccctaccaagctgaacgacctgtgcttcaccaacgtgtacgccgacagcttcgtgatcaggggcgacgaggtgaggcagatcgcccctggccagaccggcaacatcgccgactacaactacaagctgcctgacgacttcaccggctgcgtgatcgcctggaacagcaacaacctggacagcaaggtgggcggcaactacaactacctgtacaggctgttcaggaagagcaacctgaagcctttcgagagggacatcagcaccgagatctaccaggccggcagcaccccttgcaacggcgtgaagggcttcaactgctacttccctctgcagagctacggcttccagcctacctacggcgtgggctaccagccttacagggtggtggtgctgagcttcgagctgctgcacgcccctgccaccgtgtgcggccctaagaagagcaccaacctggtgaagaacaagtgcgtgaacttc

SEQ ID NO: 12 - DNA, 1617 bp, Artificial Sequence; identical to SEQ ID NO: 10
  For the preparation of mRNA vaccines

SEQ ID NO: 13 - protein, 541 aa, Artificial Sequence
  Antigen
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 14 - DNA, 1623 bp, Artificial Sequence
  For the preparation of mRNA vaccines
atgttcgtgttcctggtgctgctgcccctggtgagcagccagtgcgtgaacttcaccaacagaacccagctgcccagcgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaactaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagcgagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaccatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtgaagggcttcaactgctacttccccctgcagagctacggcttccagcccacctacggcgtgggctaccagccctacagagtggtggtgctgagcttcgagctgctgcacgcccccgccaccgtgtgcggccccaagaagagcaccaacctggtgaagaacaagtgcgtgaacttc

SEQ ID NO: 15 - DNA, 1623 bp, Artificial Sequence
  For the preparation of mRNA vaccines
atgttcgtgttcctggtgctgctgcctctggtgagcagccagtgcgtgaacttcaccaacagaacccagctgcctagcgcctacaccaacagcttcaccagaggcgtgtactaccctgacaaggtgttcagaagcagcgtgctgcacagcacccaggacctgttcctgcctttcttcagcaacgtgacctggttccacgccatccacgtgagcggcaccaacggcaccaagagattcgacaaccctgtgctgcctttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaactaccctttcctgggcgtgtactaccacaagaacaacaagagctggatggagagcgagttcagagtgtacagcagcgccaacaactgcaccttcgagtacgtgagccagcctttcctgatggacctggagggcaagcagggcaacttcaagaacctgagcgagttcgtgttcaagaacatcgacggctacttcaagatctacagcaagcacacccctatcaacctggtgagagacctgcctcagggcttcagcgccctggagcctctggtggacctgcctatcggcatcaacatcaccagattccagaccctgctggccctgcacagaagctacctgacccctggcgacagcagcagcggctggaccgccggcgccgccgcctactacgtgggctacctgcagcctagaaccttcctgctgaagtacaacgagaacggcaccatcaccgacgccgtggactgcgccctggaccctctgagcgagaccaagtgcaccctgaagagcttcaccgtggagaagggcatctaccagaccagcaacttcagagtgcagcctaccgagagcatcgtgagattccctaacatcaccaacctgtgccctttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccctaccaagctgaacgacctgtgcttcaccaacgtgtacgccgacagcttcgtgatcagaggcgacgaggtgagacagatcgcccctggccagaccggcaccatcgccgactacaactacaagctgcctgacgacttcaccggctgcgtgatcgcctggaacagcaacaacctggacagcaaggtgggcggcaactacaactacctgtacagactgttcagaaagagcaacctgaagcctttcgagagagacatcagcaccgagatctaccaggccggcagcaccccttgcaacggcgtgaagggcttcaactgctacttccctctgcagagctacggcttccagcctacctacggcgtgggctaccagccttacagagtggtggtgctgagcttcgagctgctgcacgcccctgccaccgtgtgcggccctaagaagagcaccaacctggtgaagaacaagtgcgtgaacttc

SEQ ID NO: 16 - DNA, 1623 bp, Artificial Sequence
  For the preparation of mRNA vaccines
atgttcgtgttcctggtgctgctgcccctggtgagcagccagtgcgtgaacttcaccaacaggacccagctgcccagcgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaactaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagcgagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaccatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtgaagggcttcaactgctacttccccctgcagagctacggcttccagcccacctacggcgtgggctaccagccctacagggtggtggtgctgagcttcgagctgctgcacgcccccgccaccgtgtgcggccccaagaagagcaccaacctggtgaagaacaagtgcgtgaacttc

SEQ ID NO: 17 - protein, 541 aa, Artificial Sequence
  Antigen
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 18 - DNA, 1623 bp, Artificial Sequence
  For the preparation of mRNA vaccines
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacagatacagactgttcagaaagagcaacctgaagcccttcgagagagacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcaccaacctggtgaagaacaagtgcgtgaacttc

SEQ ID NO: 19 - DNA, 1623 bp, Artificial Sequence
  For the preparation of mRNA vaccines
atgttcgtgttcctggtgctgctgcctctggtgagcagccagtgcgtgaacctgaccaccagaacccagctgcctcctgcctacaccaacagcttcaccagaggcgtgtactaccctgacaaggtgttcagaagcagcgtgctgcacagcacccaggacctgttcctgcctttcttcagcaacgtgacctggttccacgccatccacgtgagcggcaccaacggcaccaagagattcgacaaccctgtgctgcctttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccctttcctgggcgtgtactaccacaagaacaacaagagctggatggagagcgagttcagagtgtacagcagcgccaacaactgcaccttcgagtacgtgagccagcctttcctgatggacctggagggcaagcagggcaacttcaagaacctgagagagttcgtgttcaagaacatcgacggctacttcaagatctacagcaagcacacccctatcaacctggtgagagacctgcctcagggcttcagcgccctggagcctctggtggacctgcctatcggcatcaacatcaccagattccagaccctgctggccctgcacagaagctacctgacccctggcgacagcagcagcggctggaccgccggcgccgccgcctactacgtgggctacctgcagcctagaaccttcctgctgaagtacaacgagaacggcaccatcaccgacgccgtggactgcgccctggaccctctgagcgagaccaagtgcaccctgaagagcttcaccgtggagaagggcatctaccagaccagcaacttcagagtgcagcctaccgagagcatcgtgagattccctaacatcaccaacctgtgccctttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccctaccaagctgaacgacctgtgcttcaccaacgtgtacgccgacagcttcgtgatcagaggcgacgaggtgagacagatcgcccctggccagaccggcaagatcgccgactacaactacaagctgcctgacgacttcaccggctgcgtgatcgcctggaacagcaacaacctggacagcaaggtgggcggcaactacaactacagatacagactgttcagaaagagcaacctgaagcctttcgagagagacatcagcaccgagatctaccaggccggcagcaccccttgcaacggcgtggagggcttcaactgctacttccctctgcagagctacggcttccagcctaccaacggcgtgggctaccagccttacagagtggtggtgctgagcttcgagctgctgcacgcccctgccaccgtgtgcggccctaagaagagcaccaacctggtgaagaacaagtgcgtgaacttc

SEQ ID NO: 20 - DNA, 1623 bp, Artificial Sequence
  For the preparation of mRNA vaccines
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacaggtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttc

SEQ ID NO: 21 - protein, 538 aa, Artificial Sequence
  Antigen
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNF

SEQ ID NO: 22 - DNA, 1620 bp, Artificial Sequence
  For the preparation of mRNA vaccines
atgtttgtgttcctggtgctgctgcccctggtgagcagccagtgcgtgaacctgaccaccagaacacagctgcctcccgcctacaccaactccttcacccgcggcgtgtactaccctgataaggtgttcaggagctccgtgctgcacagcacccaggacctgttcctgcctttcttctctaacgtgacctggttccacgtgattagcggcacaaacggcaccaagcggttcgataaccccgtgctgcccttcaacgacggcgtgtacttcgcctccatcgaaaaaagcaacattatcagaggctggatcttcggcaccaccctggattccaagacccagtccctgctgatcgtgaacaacgccaccaacgtcgtgatcaaggtgtgtgagttccagttctgtaatgaccccttcctggaccacaagaacaacaagagctggatggagagcgagttcagggtgtacagctccgccaacaactgcacattcgagtacgtgagccagcccttcctgatggacctggagggcaagcagggcaacttcaagaatctgagagagttcgtgttcaagaacatcgatggatacttcaagatctacagcaagcacacccctatcatcgtgagggagcctgaggacctgccccagggcttcagcgccctggagcccctcgtggacctgcctatcggcatcaacatcacaaggttccagaccctgctggccctgcacaggtcctacctgacaccaggcgacagcagcagcggctggacagccggcgccgccgcctactatgtgggctacctgcagcccagaacattcctgctgaagtataacgagaatggcaccatcacagacgccgtggactgtgccctggatcccctgagcgagaccaagtgcactctgaagtcctttacagtggagaagggaatctaccagacaagcaatttcagggtgcagcctacagagagcattgtgagattccctaacatcaccaacctgtgcccctttgacgaagtgtttaacgctacacggtttgcatcagtgtatgcctggaacaggaagagaatcagtaactgtgtggccgactactctgtgctgtataacctggctcccttctttactttcaaatgctacggggtgagccctactaagctgaatgacctgtgtttcacaaacgtgtatgccgatagctttgtgatcaggggcgacgaagtgagacagatcgctccaggccagacaggcaatattgccgattacaactataaactgcccgatgacttcacaggatgcgtgattgcctggaatagcaataagctggattctaaagtgagcggcaattacaattacctgtataggctgttcagaaagagcaacctgaagcctttcgagagagacatctccactgaaatctatcaggccggcaataagccctgcaacggcgtggctggctttaattgttactttcccctgagatcctatagctttcgccccacctatggcgtgggacaccagccttacagagtggtggtgctgtccttcgagctgctgcacgcccctgccacagtgtgcggcccaaagaagtccaccaatctggtgaagaataagtgcgtgaacttctgataa

SEQ ID NO: 23 - DNA, 1614 bp, Artificial Sequence
  For the preparation of mRNA vaccines
atgttcgtgttcctggtgctgctgcctctggtgagcagccagtgcgtgaacctgaccaccaggacccagctgcctcctgcctacaccaacagcttcaccaggggcgtgtactaccctgacaaggtgttcaggagcagcgtgctgcacagcacccaggacctgttcctgcctttcttcagcaacgtgacctggttccacgtgatcagcggcaccaacggcaccaagaggttcgacaaccctgtgctgcctttcaacgacggcgtgtacttcgccagcatcgagaagagcaacatcatcaggggctggatcttcggcaccaccctggacagcaagacccagagcctgctgatcgtgaacaacgccaccaacgtggtgatcaaggtgtgcgagttccagttctgcaacgaccctttcctggaccacaagaacaacaagagctggatggagagcgagttcagggtgtacagcagcgccaacaactgcaccttcgagtacgtgagccagcctttcctgatggacctggagggcaagcagggcaacttcaagaacctgagggagttcgtgttcaagaacatcgacggctacttcaagatctacagcaagcacacccctatcctggtgagggagcctgaggacctgcctcagggcttcagcgccctggagcctctggtggacctgcctatcggcatcaacatcaccaggttccagaccctgctggccctgcacaggagctacctgacccctggcgacagcagcagcggctggaccgccggcgccgccgcctactacgtgggctacctgcagcctaggaccttcctgctgaagtacaacgagaacggcaccatcaccgacgccgtggactgcgccctggaccctctgagcgagaccaagtgcaccctgaagagcttcaccgtggagaagggcatctaccagaccagcaacttcagggtgcagcctaccgagagcatcgtgaggttccctaacatcaccaacctgtgccctttcgacgaggtgttcaacgccaccaggttcgccagcgtgtacgcctggaacaggaagaggatcagcaactgcgtggccgactacagcgtgctgtacaacctggcccctttcttcaccttcaagtgctacggcgtgagccctaccaagctgaacgacctgtgcttcaccaacgtgtacgccgacagcttcgtgatcaggggcgacgaggtgaggcagatcgcccctggccagaccggcaacatcgccgactacaactacaagctgcctgacgacttcaccggctgcgtgatcgcctggaacagcaacaagctggacagcaaggtgagcggcaactacaactacctgtacaggctgttcaggaagagcaacctgaagcctttcgagagggacatcagcaccgagatctaccaggccggcaacaagccttgcaacggcgtggccggcttcaactgctacttccctctgaggagctacagcttcaggcctacctacggcgtgggccaccagccttacagggtggtggtgctgagcttcgagctgctgcacgcccctgccaccgtgtgcggccctaagaagagcaccaacctggtgaagaacaagtgcgtgaacttc

SEQ ID NO: 24 - DNA, 1614 bp, Artificial Sequence
  For the preparation of mRNA vaccines
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgtgatcagcggcaccaacggcaccaagaggttcgacaaccccgtgctgcccttcaacgacggcgtgtacttcgccagcatcgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctggaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcctggtgagggagcccgaggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcgacgaggtgttcaacgccaccaggttcgccagcgtgtacgcctggaacaggaagaggatcagcaactgcgtggccgactacagcgtgctgtacaacctggcccccttcttcaccttcaagtgctacggcgtgagccccaccaagctgaacgacctgtgcttcaccaacgtgtacgccgacagcttcgtgatcaggggcgacgaggtgaggcagatcgcccccggccagaccggcaacatcgccgactacaactacaagctgcccgacgacttcaccggctgcgtgatcgcctggaacagcaacaagctggacagcaaggtgagcggcaactacaactacctgtacaggctgttcaggaagagcaacctgaagcccttcgagagggacatcagcaccgagatctaccaggccggcaacaagccctgcaacggcgtggccggcttcaactgctacttccccctgaggagctacagcttcaggcccacctacggcgtgggccaccagccctacagggtggtggtgctgagcttcgagctgctgcacgcccccgccaccgtgtgcggccccaagaagagcaccaacctggtgaagaacaagtgcgtgaacttc

SEQ ID NO: 25 - RNA, 1623 nt, Artificial Sequence
  mRNAvaccines
auguucguguuccuggugcugcugccccuggugagcagccagugcgugaaccugaccaccaggacccagcugccccccgccuacaccaacagcuucaccaggggcguguacuaccccgacaagguguucaggagcagcgugcugcacagcacccaggaccuguuccugcccuucuucagcaacgugaccugguuccacgccauccacgugagcggcaccaacggcaccaagagguucgacaaccccgugcugcccuucaacgacggcguguacuucgccagcaccgagaagagcaacaucaucaggggcuggaucuucggcaccacccuggacagcaagacccagagccugcugaucgugaacaacgccaccaacguggugaucaaggugugcgaguuccaguucugcaacgaccccuuccugggcguguacuaccacaagaacaacaagagcuggauggagagcgaguucaggguguacagcagcgccaacaacugcaccuucgaguacgugagccagcccuuccugauggaccuggagggcaagcagggcaacuucaagaaccugagggaguucguguucaagaacaucgacggcuacuucaagaucuacagcaagcacacccccaucaaccuggugagggaccugccccagggcuucagcgcccuggagccccugguggaccugcccaucggcaucaacaucaccagguuccagacccugcuggcccugcacaggagcuaccugacccccggcgacagcagcagcggcuggaccgccggcgccgccgccuacuacgugggcuaccugcagcccaggaccuuccugcugaaguacaacgagaacggcaccaucaccgacgccguggacugcgcccuggacccccugagcgagaccaagugcacccugaagagcuucaccguggagaagggcaucuaccagaccagcaacuucagggugcagcccaccgagagcaucgugagguuccccaacaucaccaaccugugccccuucggcgagguguucaacgccaccagguucgccagcguguacgccuggaacaggaagaggaucagcaacugcguggccgacuacagcgugcuguacaacagcgccagcuucagcaccuucaagugcuacggcgugagccccaccaagcugaacgaccugugcuucaccaacguguacgccgacagcuucgugaucaggggcgacgaggugaggcagaucgcccccggccagaccggcaagaucgccgacuacaacuacaagcugcccgacgacuucaccggcugcgugaucgccuggaacagcaacaaccuggacagcaaggugggcggcaacuacaacuaccuguacaggcuguucaggaagagcaaccugaagcccuucgagagggacaucagcaccgagaucuaccaggccggcagcacccccugcaacggcguggagggcuucaacugcuacuucccccugcagagcuacggcuuccagcccaccaacggcgugggcuaccagcccuacagggugguggugcugagcuucgagcugcugcacgcccccgccaccgugugcggccccaagaagagcaccaaccuggugaagaacaagugcgugaacuuc

SEQ ID NO: 26 - RNA, 1623 nt, Artificial Sequence
  mRNAvaccines
auguucguguuccuggugcugcugccucuggugagcagccagugcgugaaccugaccaccagaacccagcugccuccugccuacaccaacagcuucaccagaggcguguacuacccugacaagguguucagaagcagcgugcugcacagcacccaggaccuguuccugccuuucuucagcaacgugaccugguuccacgccauccacgugagcggcaccaacggcaccaagagauucgacaacccugugcugccuuucaacgacggcguguacuucgccagcaccgagaagagcaacaucaucagaggcuggaucuucggcaccacccuggacagcaagacccagagccugcugaucgugaacaacgccaccaacguggugaucaaggugugcgaguuccaguucugcaacgacccuuuccugggcguguacuaccacaagaacaacaagagcuggauggagagcgaguucagaguguacagcagcgccaacaacugcaccuucgaguacgugagccagccuuuccugauggaccuggagggcaagcagggcaacuucaagaaccugagagaguucguguucaagaacaucgacggcuacuucaagaucuacagcaagcacaccccuaucaaccuggugagagaccugccucagggcuucagcgcccuggagccucugguggaccugccuaucggcaucaacaucaccagauuccagacccugcuggcccugcacagaagcuaccugaccccuggcgacagcagcagcggcuggaccgccggcgccgccgccuacuacgugggcuaccugcagccuagaaccuuccugcugaaguacaacgagaacggcaccaucaccgacgccguggacugcgcccuggacccucugagcgagaccaagugcacccugaagagcuucaccguggagaagggcaucuaccagaccagcaacuucagagugcagccuaccgagagcaucgugagauucccuaacaucaccaaccugugcccuuucggcgagguguucaacgccaccagauucgccagcguguacgccuggaacagaaagagaaucagcaacugcguggccgacuacagcgugcuguacaacagcgccagcuucagcaccuucaagugcuacggcgugagcccuaccaagcugaacgaccugugcuucaccaacguguacgccgacagcuucgugaucagaggcgacgaggugagacagaucgccccuggccagaccggcaagaucgccgacuacaacuacaagcugccugacgacuucaccggcugcgugaucgccuggaacagcaacaaccuggacagcaaggugggcggcaacuacaacuaccuguacagacuguucagaaagagcaaccugaagccuuucgagagagacaucagcaccgagaucuaccaggccggcagcaccccuugcaacggcguggagggcuucaacugcuacuucccucugcagagcuacggcuuccagccuaccaacggcgugggcuaccagccuuacagagugguggugcugagcuucgagcugcugcacgccccugccaccgugugcggcccuaagaagagcaccaaccuggugaagaacaagugcgugaacuuc

SEQ ID NO: 27 - RNA, 1623 nt, Artificial Sequence; identical to SEQ ID NO: 25
  mRNAvaccines

SEQ ID NO: 28 - RNA, 1614 nt, Artificial Sequence
  mRNAvaccines
auguucguguuccuggugcugcugccccuggugagcagccagugcgugaaccugaccaccagaacccagcugccccccgccuacaccaacagcuucaccagaggcguguacuaccccgacaagguguucagaagcagcgugcugcacagcacccaggaccuguuccugcccuucuucagcaacgugaccugguuccacgccaucagcggcaccaacggcaccaagagauucgacaaccccgugcugcccuucaacgacggcguguacuucgccagcaccgagaagagcaacaucaucagaggcuggaucuucggcaccacccuggacagcaagacccagagccugcugaucgugaacaacgccaccaacguggugaucaaggugugcgaguuccaguucugcaacgaccccuuccugggcguguaccacaagaacaacaagagcuggauggagagcgaguucagaguguacagcagcgccaacaacugcaccuucgaguacgugagccagcccuuccugauggaccuggagggcaagcagggcaacuucaagaaccugagagaguucguguucaagaacaucgacggcuacuucaagaucuacagcaagcacacccccaucaaccuggugagagaccugccccagggcuucagcgcccuggagccccugguggaccugcccaucggcaucaacaucaccagauuccagacccugcuggcccugcacagaagcuaccugacccccggcgacagcagcagcggcuggaccgccggcgccgccgccuacuacgugggcuaccugcagcccagaaccuuccugcugaaguacaacgagaacggcaccaucaccgacgccguggacugcgcccuggacccccugagcgagaccaagugcacccugaagagcuucaccguggagaagggcaucuaccagaccagcaacuucagagugcagcccaccgagagcaucgugagauuccccaacaucaccaaccugugccccuucggcgagguguucaacgccaccagauucgccagcguguacgccuggaacagaaagagaaucagcaacugcguggccgacuacagcgugcuguacaacagcgccagcuucagcaccuucaagugcuacggcgugagccccaccaagcugaacgaccugugcuucaccaacguguacgccgacagcuucgugaucagaggcgacgaggugagacagaucgcccccggccagaccggcaagaucgccgacuacaacuacaagcugcccgacgacuucaccggcugcgugaucgccuggaacagcaacaaccuggacagcaaggugggcggcaacuacaacuaccuguacagacuguucagaaagagcaaccugaagcccuucgagagagacaucagcaccgagaucuaccaggccggcagcacccccugcaacggcguggagggcuucaacugcuacuucccccugcagagcuacggcuuccagcccaccuacggcgugggcuaccagcccuacagagugguggugcugagcuucgagcugcugcacgcccccgccaccgugugcggccccaagaagagcaccaaccuggugaagaacaagugcgugaacuuc

SEQ ID NO: 29 - RNA, 1614 nt, Artificial Sequence
  mRNAvaccines
auguucguguuccuggugcugcugccucuggugagcagccagugcgugaaccugaccaccagaacccagcugccuccugccuacaccaacagcuucaccagaggcguguacuacccugacaagguguucagaagcagcgugcugcacagcacccaggaccuguuccugccuuucuucagcaacgugaccugguuccacgccaucagcggcaccaacggcaccaagagauucgacaacccugugcugccuuucaacgacggcguguacuucgccagcaccgagaagagcaacaucaucagaggcuggaucuucggcaccacccuggacagcaagacccagagccugcugaucgugaacaacgccaccaacguggugaucaaggugugcgaguuccaguucugcaacgacccuuuccugggcguguaccacaagaacaacaagagcuggauggagagcgaguucagaguguacagcagcgccaacaacugcaccuucgaguacgugagccagccuuuccugauggaccuggagggcaagcagggcaacuucaagaaccugagagaguucguguucaagaacaucgacggcuacuucaagaucuacagcaagcacaccccuaucaaccuggugagagaccugccucagggcuucagcgcccuggagccucugguggaccugccuaucggcaucaacaucaccagauuccagacccugcuggcccugcacagaagcuaccugaccccuggcgacagcagcagcggcuggaccgccggcgccgccgccuacuacgugggcuaccugcagccuagaaccuuccugcugaaguacaacgagaacggcaccaucaccgacgccguggacugcgcccuggacccucugagcgagaccaagugcacccugaagagcuucaccguggagaagggcaucuaccagaccagcaacuucagagugcagccuaccgagagcaucgugagauucccuaacaucaccaaccugugcccuuucggcgagguguucaacgccaccagauucgccagcguguacgccuggaacagaaagagaaucagcaacugcguggccgacuacagcgugcuguacaacagcgccagcuucagcaccuucaagugcuacggcgugagcccuaccaagcugaacgaccugugcuucaccaacguguacgccgacagcuucgugaucagaggcgacgaggugagacagaucgccccuggccagaccggcaagaucgccgacuacaacuacaagcugccugacgacuucaccggcugcgugaucgccuggaacagcaacaaccuggacagcaaggugggcggcaacuacaacuaccuguacagacuguucagaaagagcaaccugaagccuuucgagagagacaucagcaccgagaucuaccaggccggcagcaccccuugcaacggcguggagggcuucaacugcuacuucccucugcagagcuacggcuuccagccuaccuacggcgugggcuaccagccuuacagagugguggugcugagcuucgagcugcugcacgccccugccaccgugugcggcccuaagaagagcaccaaccuggugaagaacaagugcgugaacuuc

SEQ ID NO: 30 - RNA, 1614 nt, Artificial Sequence
  mRNAvaccines
auguucguguuccuggugcugcugccccuggugagcagccagugcgugaaccugaccaccaggacccagcugccccccgccuacaccaacagcuucaccaggggcguguacuaccccgacaagguguucaggagcagcgugcugcacagcacccaggaccuguuccugcccuucuucagcaacgugaccugguuccacgccaucagcggcaccaacggcaccaagagguucgacaaccccgugcugcccuucaacgacggcguguacuucgccagcaccgagaagagcaacaucaucaggggcuggaucuucggcaccacccuggacagcaagacccagagccugcugaucgugaacaacgccaccaacguggugaucaaggugugcgaguuccaguucugcaacgaccccuuccugggcguguaccacaagaacaacaagagcuggauggagagcgaguucaggguguacagcagcgccaacaacugcaccuucgaguacgugagccagcccuuccugauggaccuggagggcaagcagggcaacuucaagaaccugagggaguucguguucaagaacaucgacggcuacuucaagaucuacagcaagcacacccccaucaaccuggugagggaccugccccagggcuucagcgcccuggagccccugguggaccugcccaucggcaucaacaucaccagguuccagacccugcuggcccugcacaggagcuaccugacccccggcgacagcagcagcggcuggaccgccggcgccgccgccuacuacgugggcuaccugcagcccaggaccuuccugcugaaguacaacgagaacggcaccaucaccgacgccguggacugcgcccuggacccccugagcgagaccaagugcacccugaagagcuucaccguggagaagggcaucuaccagaccagcaacuucagggugcagcccaccgagagcaucgugagguuccccaacaucaccaaccugugccccuucggcgagguguucaacgccaccagguucgccagcguguacgccuggaacaggaagaggaucagcaacugcguggccgacuacagcgugcuguacaacagcgccagcuucagcaccuucaagugcuacggcgugagccccaccaagcugaacgaccugugcuucaccaacguguacgccgacagcuucgugaucaggggcgacgaggugaggcagaucgcccccggccagaccggcaagaucgccgacuacaacuacaagcugcccgacgacuucaccggcugcgugaucgccuggaacagcaacaaccuggacagcaaggugggcggcaacuacaacuaccuguacaggcuguucaggaagagcaaccugaagcccuucgagagggacaucagcaccgagaucuaccaggccggcagcacccccugcaacggcguggagggcuucaacugcuacuucccccugcagagcuacggcuuccagcccaccuacggcgugggcuaccagcccuacagggugguggugcugagcuucgagcugcugcacgcccccgccaccgugugcggccccaagaagagcaccaaccuggugaagaacaagugcgugaacuuc

SEQ ID NO: 31 - RNA, 1617 nt, Artificial Sequence
  mRNAvaccines
auguucguguuccuggugcugcugccccuggugagcagccagugcgugaaccugaccaccaggacccagcugccccccgccuacaccaacagcuucaccaggggcguguacuaccccgacaagguguucaggagcagcgugcugcacagcacccaggaccuguuccugcccuucuucagcaacgugaccugguuccacgccauccacgugagcggcaccaacggcaccaagagguucgccaaccccgugcugcccuucaacgacggcguguacuucgccagcaccgagaagagcaacaucaucaggggcuggaucuucggcaccacccuggacagcaagacccagagccugcugaucgugaacaacgccaccaacguggugaucaaggugugcgaguuccaguucugcaacgaccccuuccugggcguguacuaccacaagaacaacaagagcuggauggagagcgaguucaggguguacagcagcgccaacaacugcaccuucgaguacgugagccagcccuuccugauggaccuggagggcaagcagggcaacuucaagaaccugagggaguucguguucaagaacaucgacggcuacuucaagaucuacagcaagcacacccccaucaaccuggugaggggccugccccagggcuucagcgcccuggagccccugguggaccugcccaucggcaucaacaucaccagguuccagacccugcugcacaggagcuaccugacccccggcgacagcagcagcggcuggaccgccggcgccgccgccuacuacgugggcuaccugcagcccaggaccuuccugcugaaguacaacgagaacggcaccaucaccgacgccguggacugcgcccuggacccccugagcgagaccaagugcacccugaagagcuucaccguggagaagggcaucuaccagaccagcaacuucagggugcagcccaccgagagcaucgugagguuccccaacaucaccaaccugugccccuucggcgagguguucaacgccaccagguucgccagcguguacgccuggaacaggaagaggaucagcaacugcguggccgacuacagcgugcuguacaacagcgccagcuucagcaccuucaagugcuacggcgugagccccaccaagcugaacgaccugugcuucaccaacguguacgccgacagcuucgugaucaggggcgacgaggugaggcagaucgcccccggccagaccggcaacaucgccgacuacaacuacaagcugcccgacgacuucaccggcugcgugaucgccuggaacagcaacaaccuggacagcaaggugggcggcaacuacaacuaccuguacaggcuguucaggaagagcaaccugaagcccuucgagagggacaucagcaccgagaucuaccaggccggcagcacccccugcaacggcgugaagggcuucaacugcuacuucccccugcagagcuacggcuuccagcccaccuacggcgugggcuaccagcccuacagggugguggugcugagcuucgagcugcugcacgcccccgccaccgugugcggccccaagaagagcaccaaccuggugaagaacaagugcgugaacuuc

SEQ ID NO: 32 - RNA, 1617 nt, Artificial Sequence
  mRNAvaccines
auguucguguuccuggugcugcugccucuggugagcagccagugcgugaaccugaccaccaggacccagcugccuccugccuacaccaacagcuucaccaggggcguguacuacccugacaagguguucaggagcagcgugcugcacagcacccaggaccuguuccugccuuucuucagcaacgugaccugguuccacgccauccacgugagcggcaccaacggcaccaagagguucgccaacccugugcugccuuucaacgacggcguguacuucgccagcaccgagaagagcaacaucaucaggggcuggaucuucggcaccacccuggacagcaagacccagagccugcugaucgugaacaacgccaccaacguggugaucaaggugugcgaguuccaguucugcaacgacccuuuccugggcguguacuaccacaagaacaacaagagcuggauggagagcgaguucaggguguacagcagcgccaacaacugcaccuucgaguacgugagccagccuuuccugauggaccuggagggcaagcagggcaacuucaagaaccugagggaguucguguucaagaacaucgacggcuacuucaagaucuacagcaagcacaccccuaucaaccuggugaggggccugccucagggcuucagcgcccuggagccucugguggaccugccuaucggcaucaacaucaccagguuccagacccugcugcacaggagcuaccugaccccuggcgacagcagcagcggcuggaccgccggcgccgccgccuacuacgugggcuaccugcagccuaggaccuuccugcugaaguacaacgagaacggcaccaucaccgacgccguggacugcgcccuggacccucugagcgagaccaagugcacccugaagagcuucaccguggagaagggcaucuaccagaccagcaacuucagggugcagccuaccgagagcaucgugagguucccuaacaucaccaaccugugcccuuucggcgagguguucaacgccaccagguucgccagcguguacgccuggaacaggaagaggaucagcaacugcguggccgacuacagcgugcuguacaacagcgccagcuucagcaccuucaagugcuacggcgugagcccuaccaagcugaacgaccugugcuucaccaacguguacgccgacagcuucgugaucaggggcgacgaggugaggcagaucgccccuggccagaccggcaacaucgccgacuacaacuacaagcugccugacgacuucaccggcugcgugaucgccuggaacagcaacaaccuggacagcaaggugggcggcaacuacaacuaccuguacaggcuguucaggaagagcaaccugaagccuuucgagagggacaucagcaccgagaucuaccaggccggcagcaccccuugcaacggcgugaagggcuucaacugcuacuucccucugcagagcuacggcuuccagccuaccuacggcgugggcuaccagccuuacagggugguggugcugagcuucgagcugcugcacgccccugccaccgugugcggcccuaagaagagcaccaaccuggugaagaacaagugcgugaacuuc

SEQ ID NO: 33 - RNA, 1617 nt, Artificial Sequence; identical to SEQ ID NO: 31
  mRNAvaccines

SEQ ID NO: 34 - RNA, 1623 nt, Artificial Sequence
  mRNAvaccines
auguucguguuccuggugcugcugccccuggugagcagccagugcgugaacuucaccaacagaacccagcugcccagcgccuacaccaacagcuucaccagaggcguguacuaccccgacaagguguucagaagcagcgugcugcacagcacccaggaccuguuccugcccuucuucagcaacgugaccugguuccacgccauccacgugagcggcaccaacggcaccaagagauucgacaaccccgugcugcccuucaacgacggcguguacuucgccagcaccgagaagagcaacaucaucagaggcuggaucuucggcaccacccuggacagcaagacccagagccugcugaucgugaacaacgccaccaacguggugaucaaggugugcgaguuccaguucugcaacuaccccuuccugggcguguacuaccacaagaacaacaagagcuggauggagagcgaguucagaguguacagcagcgccaacaacugcaccuucgaguacgugagccagcccuuccugauggaccuggagggcaagcagggcaacuucaagaaccugagcgaguucguguucaagaacaucgacggcuacuucaagaucuacagcaagcacacccccaucaaccuggugagagaccugccccagggcuucagcgcccuggagccccugguggaccugcccaucggcaucaacaucaccagauuccagacccugcuggcccugcacagaagcuaccugacccccggcgacagcagcagcggcuggaccgccggcgccgccgccuacuacgugggcuaccugcagcccagaaccuuccugcugaaguacaacgagaacggcaccaucaccgacgccguggacugcgcccuggacccccugagcgagaccaagugcacccugaagagcuucaccguggagaagggcaucuaccagaccagcaacuucagagugcagcccaccgagagcaucgugagauuccccaacaucaccaaccugugccccuucggcgagguguucaacgccaccagauucgccagcguguacgccuggaacagaaagagaaucagcaacugcguggccgacuacagcgugcuguacaacagcgccagcuucagcaccuucaagugcuacggcgugagccccaccaagcugaacgaccugugcuucaccaacguguacgccgacagcuucgugaucagaggcgacgaggugagacagaucgcccccggccagaccggcaccaucgccgacuacaacuacaagcugcccgacgacuucaccggcugcgugaucgccuggaacagcaacaaccuggacagcaaggugggcggcaacuacaacuaccuguacagacuguucagaaagagcaaccugaagcccuucgagagagacaucagcaccgagaucuaccaggccggcagcacccccugcaacggcgugaagggcuucaacugcuacuucccccugcagagcuacggcuuccagcccaccuacggcgugggcuaccagcccuacagagugguggugcugagcuucgagcugcugcacgcccccgccaccgugugcggccccaagaagagcaccaaccuggugaagaacaagugcgugaacuuc

SEQ ID NO: 35 - RNA, 1623 nt, Artificial Sequence
  mRNAvaccines
auguucguguuccuggugcugcugccucuggugagcagccagugcgugaacuucaccaacagaacccagcugccuagcgccuacaccaacagcuucaccagaggcguguacuacccugacaagguguucagaagcagcgugcugcacagcacccaggaccuguuccugccuuucuucagcaacgugaccugguuccacgccauccacgugagcggcaccaacggcaccaagagauucgacaacccugugcugccuuucaacgacggcguguacuucgccagcaccgagaagagcaacaucaucagaggcuggaucuucggcaccacccuggacagcaagacccagagccugcugaucgugaacaacgccaccaacguggugaucaaggugugcgaguuccaguucugcaacuacccuuuccugggcguguacuaccacaagaacaacaagagcuggauggagagcgaguucagaguguacagcagcgccaacaacugcaccuucgaguacgugagccagccuuuccugauggaccuggagggcaagcagggcaacuucaagaaccugagcgaguucguguucaagaacaucgacggcuacuucaagaucuacagcaagcacaccccuaucaaccuggugagagaccugccucagggcuucagcgcccuggagccucugguggaccugccuaucggcaucaacaucaccagauuccagacccugcuggcccugcacagaagcuaccugaccccuggcgacagcagcagcggcuggaccgccggcgccgccgccuacuacgugggcuaccugcagccuagaaccuuccugcugaaguacaacgagaacggcaccaucaccgacgccguggacugcgcccuggacccucugagcgagaccaagugcacccugaagagcuucaccguggagaagggcaucuaccagaccagcaacuucagagugcagccuaccgagagcaucgugagauucccuaacaucaccaaccugugcccuuucggcgagguguucaacgccaccagauucgccagcguguacgccuggaacagaaagagaaucagcaacugcguggccgacuacagcgugcuguacaacagcgccagcuucagcaccuucaagugcuacggcgugagcccuaccaagcugaacgaccugugcuucaccaacguguacgccgacagcuucgugaucagaggcgacgaggugagacagaucgccccuggccagaccggcaccaucgccgacuacaacuacaagcugccugacgacuucaccggcugcgugaucgccuggaacagcaacaaccuggacagcaaggugggcggcaacuacaacuaccuguacagacuguucagaaagagcaaccugaagccuuucgagagagacaucagcaccgagaucuaccaggccggcagcaccccuugcaacggcgugaagggcuucaacugcuacuucccucugcagagcuacggcuuccagccuaccuacggcgugggcuaccagccuuacagagugguggugcugagcuucgagcugcugcacgccccugccaccgugugcggcccuaagaagagcaccaaccuggugaagaacaagugcgugaacuuc

SEQ ID NO: 36 - RNA, 1623 nt, Artificial Sequence
  mRNAvaccines
auguucguguuccuggugcugcugccccuggugagcagccagugcgugaacuucaccaacaggacccagcugcccagcgccuacaccaacagcuucaccaggggcguguacuaccccgacaagguguucaggagcagcgugcugcacagcacccaggaccuguuccugcccuucuucagcaacgugaccugguuccacgccauccacgugagcggcaccaacggcaccaagagguucgacaaccccgugcugcccuucaacgacggcguguacuucgccagcaccgagaagagcaacaucaucaggggcuggaucuucggcaccacccuggacagcaagacccagagccugcugaucgugaacaacgccaccaacguggugaucaaggugugcgaguuccaguucugcaacuaccccuuccugggcguguacuaccacaagaacaacaagagcuggauggagagcgaguucaggguguacagcagcgccaacaacugcaccuucgaguacgugagccagcccuuccugauggaccuggagggcaagcagggcaacuucaagaaccugagcgaguucguguucaagaacaucgacggcuacuucaagaucuacagcaagcacacccccaucaaccuggugagggaccugccccagggcuucagcgcccuggagccccugguggaccugcccaucggcaucaacaucaccagguuccagacccugcuggcccugcacaggagcuaccugacccccggcgacagcagcagcggcuggaccgccggcgccgccgccuacuacgugggcuaccugcagcccaggaccuuccugcugaaguacaacgagaacggcaccaucaccgacgccguggacugcgcccuggacccccugagcgagaccaagugcacccugaagagcuucaccguggagaagggcaucuaccagaccagcaacuucagggugcagcccaccgagagcaucgugagguuccccaacaucaccaaccugugccccuucggcgagguguucaacgccaccagguucgccagcguguacgccuggaacaggaagaggaucagcaacugcguggccgacuacagcgugcuguacaacagcgccagcuucagcaccuucaagugcuacggcgugagccccaccaagcugaacgaccugugcuucaccaacguguacgccgacagcuucgugaucaggggcgacgaggugaggcagaucgcccccggccagaccggcaccaucgccgacuacaacuacaagcugcccgacgacuucaccggcugcgugaucgccuggaacagcaacaaccuggacagcaaggugggcggcaacuacaacuaccuguacaggcuguucaggaagagcaaccugaagcccuucgagagggacaucagcaccgagaucuaccaggccggcagcacccccugcaacggcgugaagggcuucaacugcuacuucccccugcagagcuacggcuuccagcccaccuacggcgugggcuaccagcccuacagggugguggugcugagcuucgagcugcugcacgcccccgccaccgugugcggccccaagaagagcaccaaccuggugaagaacaagugcgugaacuuc

SEQ ID NO: 37 - RNA, 1623 nt, Artificial Sequence
  mRNAvaccines
auguucguguuccuggugcugcugccccuggugagcagccagugcgugaaccugaccaccagaacccagcugccccccgccuacaccaacagcuucaccagaggcguguacuaccccgacaagguguucagaagcagcgugcugcacagcacccaggaccuguuccugcccuucuucagcaacgugaccugguuccacgccauccacgugagcggcaccaacggcaccaagagauucgacaaccccgugcugcccuucaacgacggcguguacuucgccagcaccgagaagagcaacaucaucagaggcuggaucuucggcaccacccuggacagcaagacccagagccugcugaucgugaacaacgccaccaacguggugaucaaggugugcgaguuccaguucugcaacgaccccuuccugggcguguacuaccacaagaacaacaagagcuggauggagagcgaguucagaguguacagcagcgccaacaacugcaccuucgaguacgugagccagcccuuccugauggaccuggagggcaagcagggcaacuucaagaaccugagagaguucguguucaagaacaucgacggcuacuucaagaucuacagcaagcacacccccaucaaccuggugagagaccugccccagggcuucagcgcccuggagccccugguggaccugcccaucggcaucaacaucaccagauuccagacccugcuggcccugcacagaagcuaccugacccccggcgacagcagcagcggcuggaccgccggcgccgccgccuacuacgugggcuaccugcagcccagaaccuuccugcugaaguacaacgagaacggcaccaucaccgacgccguggacugcgcccuggacccccugagcgagaccaagugcacccugaagagcuucaccguggagaagggcaucuaccagaccagcaacuucagagugcagcccaccgagagcaucgugagauuccccaacaucaccaaccugugccccuucggcgagguguucaacgccaccagauucgccagcguguacgccuggaacagaaagagaaucagcaacugcguggccgacuacagcgugcuguacaacagcgccagcuucagcaccuucaagugcuacggcgugagccccaccaagcugaacgaccugugcuucaccaacguguacgccgacagcuucgugaucagaggcgacgaggugagacagaucgcccccggccagaccggcaagaucgccgacuacaacuacaagcugcccgacgacuucaccggcugcgugaucgccuggaacagcaacaaccuggacagcaaggugggcggcaacuacaacuacagauacagacuguucagaaagagcaaccugaagcccuucgagagagacaucagcaccgagaucuaccaggccggcagcacccccugcaacggcguggagggcuucaacugcuacuucccccugcagagcuacggcuuccagcccaccaacggcgugggcuaccagcccuacagagugguggugcugagcuucgagcugcugcacgcccccgccaccgugugcggccccaagaagagcaccaaccuggugaagaacaagugcgugaacuuc

SEQ ID NO: 38 - RNA, 1623 nt, Artificial Sequence
  mRNAvaccines
auguucguguuccuggugcugcugccucuggugagcagccagugcgugaaccugaccaccagaacccagcugccuccugccuacaccaacagcuucaccagaggcguguacuacccugacaagguguucagaagcagcgugcugcacagcacccaggaccuguuccugccuuucuucagcaacgugaccugguuccacgccauccacgugagcggcaccaacggcaccaagagauucgacaacccugugcugccuuucaacgacggcguguacuucgccagcaccgagaagagcaacaucaucagaggcuggaucuucggcaccacccuggacagcaagacccagagccugcugaucgugaacaacgccaccaacguggugaucaaggugugcgaguuccaguucugcaacgacccuuuccugggcguguacuaccacaagaacaacaagagcuggauggagagcgaguucagaguguacagcagcgccaacaacugcaccuucgaguacgugagccagccuuuccugauggaccuggagggcaagcagggcaacuucaagaaccugagagaguucguguucaagaacaucgacggcuacuucaagaucuacagcaagcacaccccuaucaaccuggugagagaccugccucagggcuucagcgcccuggagccucugguggaccugccuaucggcaucaacaucaccagauuccagacccugcuggcccugcacagaagcuaccugaccccuggcgacagcagcagcggcuggaccgccggcgccgccgccuacuacgugggcuaccugcagccuagaaccuuccugcugaaguacaacgagaacggcaccaucaccgacgccguggacugcgcccuggacccucugagcgagaccaagugcacccugaagagcuucaccguggagaagggcaucuaccagaccagcaacuucagagugcagccuaccgagagcaucgugagauucccuaacaucaccaaccugugcccuuucggcgagguguucaacgccaccagauucgccagcguguacgccuggaacagaaagagaaucagcaacugcguggccgacuacagcgugcuguacaacagcgccagcuucagcaccuucaagugcuacggcgugagcccuaccaagcugaacgaccugugcuucaccaacguguacgccgacagcuucgugaucagaggcgacgaggugagacagaucgccccuggccagaccggcaagaucgccgacuacaacuacaagcugccugacgacuucaccggcugcgugaucgccuggaacagcaacaaccuggacagcaaggugggcggcaacuacaacuacagauacagacuguucagaaagagcaaccugaagccuuucgagagagacaucagcaccgagaucuaccaggccggcagcaccccuugcaacggcguggagggcuucaacugcuacuucccucugcagagcuacggcuuccagccuaccaacggcgugggcuaccagccuuacagagugguggugcugagcuucgagcugcugcacgccccugccaccgugugcggcccuaagaagagcaccaaccuggugaagaacaagugcgugaacuuc

SEQ ID NO: 39 - RNA, 1623 nt, Artificial Sequence
  mRNAvaccines
auguucguguuccuggugcugcugccccuggugagcagccagugcgugaaccugaccaccaggacccagcugccccccgccuacaccaacagcuucaccaggggcguguacuaccccgacaagguguucaggagcagcgugcugcacagcacccaggaccuguuccugcccuucuucagcaacgugaccugguuccacgccauccacgugagcggcaccaacggcaccaagagguucgacaaccccgugcugcccuucaacgacggcguguacuucgccagcaccgagaagagcaacaucaucaggggcuggaucuucggcaccacccuggacagcaagacccagagccugcugaucgugaacaacgccaccaacguggugaucaaggugugcgaguuccaguucugcaacgaccccuuccugggcguguacuaccacaagaacaacaagagcuggauggagagcgaguucaggguguacagcagcgccaacaacugcaccuucgaguacgugagccagcccuuccugauggaccuggagggcaagcagggcaacuucaagaaccugagggaguucguguucaagaacaucgacggcuacuucaagaucuacagcaagcacacccccaucaaccuggugagggaccugccccagggcuucagcgcccuggagccccugguggaccugcccaucggcaucaacaucaccagguuccagacccugcuggcccugcacaggagcuaccugacccccggcgacagcagcagcggcuggaccgccggcgccgccgccuacuacgugggcuaccugcagcccaggaccuuccugcugaaguacaacgagaacggcaccaucaccgacgccguggacugcgcccuggacccccugagcgagaccaagugcacccugaagagcuucaccguggagaagggcaucuaccagaccagcaacuucagggugcagcccaccgagagcaucgugagguuccccaacaucaccaaccugugccccuucggcgagguguucaacgccaccagguucgccagcguguacgccuggaacaggaagaggaucagcaacugcguggccgacuacagcgugcuguacaacagcgccagcuucagcaccuucaagugcuacggcgugagccccaccaagcugaacgaccugugcuucaccaacguguacgccgacagcuucgugaucaggggcgacgaggugaggcagaucgcccccggccagaccggcaagaucgccgacuacaacuacaagcugcccgacgacuucaccggcugcgugaucgccuggaacagcaacaaccuggacagcaaggugggcggcaacuacaacuacagguacaggcuguucaggaagagcaaccugaagcccuucgagagggacaucagcaccgagaucuaccaggccggcagcacccccugcaacggcguggagggcuucaacugcuacuucccccugcagagcuacggcuuccagcccaccaacggcgugggcuaccagcccuacagggugguggugcugagcuucgagcugcugcacgcccccgccaccgugugcggccccaagaagagcaccaaccuggugaagaacaagugcgugaacuuc

SEQ ID NO: 40 - RNA, 1620 nt, Artificial Sequence
  mRNAvaccines
auguuuguguuccuggugcugcugccccuggugagcagccagugcgugaaccugaccaccagaacacagcugccucccgccuacaccaacuccuucacccgcggcguguacuacccugauaagguguucaggagcuccgugcugcacagcacccaggaccuguuccugccuuucuucucuaacgugaccugguuccacgugauuagcggcacaaacggcaccaagcgguucgauaaccccgugcugcccuucaacgacggcguguacuucgccuccaucgaaaaaagcaacauuaucagaggcuggaucuucggcaccacccuggauuccaagacccagucccugcugaucgugaacaacgccaccaacgucgugaucaaggugugugaguuccaguucuguaaugaccccuuccuggaccacaagaacaacaagagcuggauggagagcgaguucaggguguacagcuccgccaacaacugcacauucgaguacgugagccagcccuuccugauggaccuggagggcaagcagggcaacuucaagaaucugagagaguucguguucaagaacaucgauggauacuucaagaucuacagcaagcacaccccuaucaucgugagggagccugaggaccugccccagggcuucagcgcccuggagccccucguggaccugccuaucggcaucaacaucacaagguuccagacccugcuggcccugcacagguccuaccugacaccaggcgacagcagcagcggcuggacagccggcgccgccgccuacuaugugggcuaccugcagcccagaacauuccugcugaaguauaacgagaauggcaccaucacagacgccguggacugugcccuggauccccugagcgagaccaagugcacucugaaguccuuuacaguggagaagggaaucuaccagacaagcaauuucagggugcagccuacagagagcauugugagauucccuaacaucaccaaccugugccccuuugacgaaguguuuaacgcuacacgguuugcaucaguguaugccuggaacaggaagagaaucaguaacuguguggccgacuacucugugcuguauaaccuggcucccuucuuuacuuucaaaugcuacggggugagcccuacuaagcugaaugaccuguguuucacaaacguguaugccgauagcuuugugaucaggggcgacgaagugagacagaucgcuccaggccagacaggcaauauugccgauuacaacuauaaacugcccgaugacuucacaggaugcgugauugccuggaauagcaauaagcuggauucuaaagugagcggcaauuacaauuaccuguauaggcuguucagaaagagcaaccugaagccuuucgagagagacaucuccacugaaaucuaucaggccggcaauaagcccugcaacggcguggcuggcuuuaauuguuacuuuccccugagauccuauagcuuucgccccaccuauggcgugggacaccagccuuacagagugguggugcuguccuucgagcugcugcacgccccugccacagugugcggcccaaagaaguccaccaaucuggugaagaauaagugcgugaacuucugauaa

SEQ ID NO: 41 - RNA, 1614 nt, Artificial Sequence
  mRNAvaccines
auguucguguuccuggugcugcugccccuggugagcagccagugcgugaaccugaccaccaggacccagcugccccccgccuacaccaacagcuucaccaggggcguguacuaccccgacaagguguucaggagcagcgugcugcacagcacccaggaccuguuccugcccuucuucagcaacgugaccugguuccacgugaucagcggcaccaacggcaccaagagguucgacaaccccgugcugcccuucaacgacggcguguacuucgccagcaucgagaagagcaacaucaucaggggcuggaucuucggcaccacccuggacagcaagacccagagccugcugaucgugaacaacgccaccaacguggugaucaaggugugcgaguuccaguucugcaacgaccccuuccuggaccacaagaacaacaagagcuggauggagagcgaguucaggguguacagcagcgccaacaacugcaccuucgaguacgugagccagcccuuccugauggaccuggagggcaagcagggcaacuucaagaaccugagggaguucguguucaagaacaucgacggcuacuucaagaucuacagcaagcacacccccauccuggugagggagcccgaggaccugccccagggcuucagcgcccuggagccccugguggaccugcccaucggcaucaacaucaccagguuccagacccugcuggcccugcacaggagcuaccugacccccggcgacagcagcagcggcuggaccgccggcgccgccgccuacuacgugggcuaccugcagcccaggaccuuccugcugaaguacaacgagaacggcaccaucaccgacgccguggacugcgcccuggacccccugagcgagaccaagugcacccugaagagcuucaccguggagaagggcaucuaccagaccagcaacuucagggugcagcccaccgagagcaucgugagguuccccaacaucaccaaccugugccccuucgacgagguguucaacgccaccagguucgccagcguguacgccuggaacaggaagaggaucagcaacugcguggccgacuacagcgugcuguacaaccuggcccccuucuucaccuucaagugcuacggcgugagccccaccaagcugaacgaccugugcuucaccaacguguacgccgacagcuucgugaucaggggcgacgaggugaggcagaucgcccccggccagaccggcaacaucgccgacuacaacuacaagcugcccgacgacuucaccggcugcgugaucgccuggaacagcaacaagcuggacagcaaggugagcggcaacuacaacuaccuguacaggcuguucaggaagagcaaccugaagcccuucgagagggacaucagcaccgagaucuaccaggccggcaacaagcccugcaacggcguggccggcuucaacugcuacuucccccugaggagcuacagcuucaggcccaccuacggcgugggccaccagcccuacagggugguggugcugagcuucgagcugcugcacgcccccgccaccgugugcggccccaagaagagcaccaaccuggugaagaacaagugcgugaacuuc

SEQ ID NO: 42 - RNA, 1614 nt, Artificial Sequence; identical to SEQ ID NO: 41
  mRNAvaccines

SEQ ID NO: 43 - RNA, 672 nt, Artificial Sequence
  mRNAvaccines
augagggugcagcccaccgagagcaucgugagguuccccaacaucaccaaccugugccccuucggcgagguguucaacgccaccagguucgccagcguguacgccuggaacaggaagaggaucagcaacugcguggccgacuacagcgugcuguacaacagcgccagcuucagcaccuucaagugcuacggcgugagccccaccaagcugaacgaccugugcuucaccaacguguacgccgacagcuucgugaucaggggcgacgaggugaggcagaucgcccccggccagaccggcaagaucgccgacuacaacuacaagcugcccgacgacuucaccggcugcgugaucgccuggaacagcaacaaccuggacagcaaggugggcggcaacuacaacuaccuguacaggcuguucaggaagagcaaccugaagcccuucgagagggacaucagcaccgagaucuaccaggccggcagcacccccugcaacggcguggagggcuucaacugcuacuucccccugcagagcuacggcuuccagcccaccaacggcgugggcuaccagcccuacagggugguggugcugagcuucgagcugcugcacgcccccgccaccgugugcggccccaagaagagcaccaaccuggugaagaacaagugcgugaacuuc

SEQ ID NO: 44 - protein, 529 aa, Artificial Sequence
  NTD-RBD
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 45 - protein, 526 aa, Artificial Sequence
  NTD-RBD
SQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNF

SEQ ID NO: 46 - protein, 527 aa, Artificial Sequence
  NTD-RBD
SQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 47 - protein, 529 aa, Artificial Sequence
  NTD-RBD
SQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 48 - protein, 529 aa, Artificial Sequence
  NTD-RBD
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 49 - protein, 526 aa, Artificial Sequence
  NTD-RBD
SQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNF

SEQ ID NO: 50 - protein, 14 aa, Artificial Sequence
  linking peptide
SFTVEKGIYQTSNF

SEQ ID NO: 51 - RNA, 51 nt, Artificial Sequence
  5′UTR
agggagauaagagagaaaagaagaguaagaagaaauauaagagccgccacc

SEQ ID NO: 52 - RNA, 93 nt, Artificial Sequence
  3′UTR
gcugccuucugcggggcuugccuucuggccaugcccuucuucucucccuugcaccuguaccucuuggucuuugaauaaagccugaguaggaag

SEQ ID NO: 53 - RNA, 112 nt, Artificial Sequence
  Poly-A tail
aaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaaa

SEQ ID NO: 54 - RNA, 1614 nt, Artificial Sequence
  mRNAvaccines
auguucguguuccuggugcugcugccguuagugaguagucagugugugaaccugacuacucgcacgcagcugccaccagcguacacgaacagcuucacgcggggcguguauuaucccgacaaaguauuucgcagcagcguccugcacuccacccaggaccucuuccucccguucuucuccaaugugacgugguuucacgucauaucggggacgaacgggacgaagagguucgacaauccgguccugccuuuuaacgacgggguguauuucgcgucaauagagaagaguaauauuauccgaggguggaucuucgggaccacccuggauaguaagacucagucucuauugaucgugaauaacgccaccaacgucguuauaaaggucugcgaguuucaguucugcaaugauccauucuuggaucauaagaacaauaagucguggauggagagcgaguuucgaguguacucgagugcaaacaauugcacuuucgaguacgucucgcagccguucuugauggaccuggaggguaagcaggggaauuucaagaaucuuagagaauucgucuuuaagaauauugacggauacuuuaagauuuauuccaagcauaccccuauuaucguccgggagccggaggaccugccgcagggcuucagcgcucuggagccgcugguugaccucccgauagggaucaacaucacgcgguuccagacgcuguuagcccugcacagguccuaccugacucccggcgauaguaguucuggguggacugcaggcgcugcugcguacuaugucggguacuuacagccccgcacguucuuguugaaguacaacgagaacggcacaaucacggacgccguugauugugcccuagauccguuaucggagaccaaguguacucucaagucguucaccguggagaagggcaucuaccagacguccaacuucagggugcagccgacugagaguauaguucgguuuccgaacauaacgaaucuaugcccguuugacgagguguucaaugcgacgcgcuucgccuccguguaugcauggaaccgaaaacgcauaagcaauugugucgcagauuauucaguuuuguacaaucucgccccguucuucacguucaaguguuacggggugaguccgacaaagcugaaugaucuguguuucacaaauguuuaugcggauucguucgugauacgcggagacgaagugcgucagauugcuccuggucagacgggcaauauagccgacuauaacuacaagcuaccagacgacuucacugguugugugauagcuuggaauucgaacaagcuggacagcaagguaucagguaauuacaauuaccuguaccggcuguucaggaagucgaauuugaagccuuucgagcgcgacaucucgaccgagaucuaccaggccggaaacaagccgugcaacggaguagcaggcuucaacugcuacuuuccguugcgcagcuacaguuuccggccuaccuacgggguagggcaccagcccuaccgcguaguggugcucuccuucgagcugcuccacgcaccggccacggugugugggccgaagaagagcaccaaucuggucaagaacaagugcgugaacuuu

SEQ ID NO: 55 - RNA, 1614 nt, Artificial Sequence
  mRNAvaccines
auguucgucuuccuggugcugcuuccgcuggucaguucacagugugugaacuugaccacgcggacgcagcuaccccccgcguauacgaacucguuuacgcgggggguauauuacccggauaagguguuucguaguuccguucuccacaguacucaggaccucuuccucccguucuucuccaaugugacgugguuucacgucauaucggggacgaacgggacgaagagguucgacaaccccguguugcccuucaacgacgggguuuauuucgcgucaauagagaagaguaauauuauccgaggguggaucuucgggaccacccuggauaguaagacucagucucuauugaucgugaacaacgccacuaauguggugaucaaggugugcgaguuucaguucugcaaugauccauucuuggaucauaagaacaacaagaguuggauggagagugaauuccguguuuacucuucagccaacaauuguaccuucgaguaugucagucagccguuucugauggaucuugaaggcaagcagggcaauuucaagaauuugcgcgaguuugucuucaagaacaucgacggcuacuucaagauauacucgaagcacacgccgauaauugugcgcgagccggaagaucuaccacagggcuucagugcucuggagcccuugguagaucuuccgaucggcauaaauaucacgcguuuccagacgcugcuggcguugcaccggaguuaccuuacuccgggcgacuccagcagcggcuggacggcaggggcugcagcguacuauguaggguaucugcaaccgcgcacguucuuguugaaguacaacgagaacggcacgauuacagaugccguagauugugcgcuggacccccugucggagacuaagugcaccuugaagaguuuuacuguggagaagggaaucuaccagacaucuaacuuccgggugcagccgacagagaguauuguacguuucccgaacaucacgaaucuaugcccguuugacgagguguuuaacgccacgcgguuugcuaguguguaugcguggaaucgcaagcgcauuagcaacugcguggcggauuauucaguuuuguauaaucucgccccguucuucacguucaaguguuacggggugaguccuacaaagcugaaugaucucugcuucaccaacgucuacgcggauagcuuuguuauccgcggagacgaggugaggcagauagcaccuggucagacgggcaauauugcugacuacaacuauaagcucccggaugacuucaccgggugcguuauagcguggaacagcaauaaguuagauucgaaggugucgggaaacuacaauuaucucuaucggcuguucaggaagucgaacuuaaagccuuuugagcgugauauaucuaccgagauauaucaggcugggaauaagccgugcaacgggguagcaggcuucaacugcuacuucccguugcgcagcuauuccuuucggcccaccuacgggguagggcaccagcccuaccgcguaguuguacugucguuugagcugcugcaugcuccagcgacaguaugugggccgaagaagucgacgaacuuggugaagaacaagugcgucaacuuc